(19) United States
(12) Patent Application Publication
(10) Pub. No.: US 2024/0221159 A1
(43) Pub. Date: Jul. 4, 2024

(54) SYSTEMS AND METHODS FOR IDENTIFICATION OF PANCREATIC DUCTAL ADENOCARCINOMA MOLECULAR SUBTYPES

(71) Applicant: OWKIN, INC., New York, NY (US)

(72) Inventors: Charles SAILLARD, Paris (FR); Benoit SCHMAUCH, Paris (FR); Victor AUBERT, Paris (FR); Kamoun AURÉLIE, Paris (FR); Magali LACROIX-TRIKI, Paris (FR); Ingrid GARBERIS, Paris (FR); Damien DRUBAY, Paris (FR); Fabrice ANDRÉ, Paris (FR); Jérôme CROS, Clichy (FR)

(21) Appl. No.: 18/558,519

(22) PCT Filed: May 6, 2022
(86) PCT No.: PCT/US2022/028140

§ 371 Date: Nov. 1, 2023

(30) Foreign Application Priority Data
May 7, 2021 (EP) 21305595.7
May 19, 2021 (EP) 21305656.7
Nov. 17, 2021 (EP) 21306599.8

(51) Int. Cl. G06T 7/00; G16H 50/20

(52) U.S. Cl. CPC G06T 7/0012; G16H 50/20; G06T 2207/20081; G06T 2207/30024

(57) ABSTRACT

Deep learning models for predicting one or more features of pancreatic ductal adenocarcinoma from histopathology slide images is provided.

### CROSS-REFERENCE TO RELATED APPLICATION

This application claims priority to European Patent Application Numbers EP21305595.7, filed May 7, 2021: EP21305656.7, filed May 19, 2021: and EP21306599.8, filed on Nov. 17, 2021. The entire content of the foregoing priority application is incorporated herein by reference.

### FIELD OF INVENTION

This invention relates generally to machine learning and computer vision and more particularly to image preprocessing and classification.

### BACKGROUND OF THE INVENTION

Histopathological image analysis (HIA) is a critical element of diagnosis in many areas of medicine, and especially in oncology. Pancreatic ductal adenocarcinoma (abbreviated “PAC” or “PDA”) is predicted to be the second cause of death by cancer in 2030, and its prognosis has seen little improvement in the last decades. PAC is a very heterogeneous tumor with preeminent stroma and multiple histological aspects. Genomic and proteomic studies have confirmed the molecular heterogeneity of PAC, and is possibly one of the factors explaining the failure of most clinical trials. Transcriptomic subtypes of PAC have been described with major prognostic and predictive implications. For example, Rashid et al., Clinical Cancer Research (2020): 26:82-92, described a single-sample classifier for PAC subtyping named Purity Independent Subtyping of Tumors (PurIST), which is based on gene expression data derived from RNAseq, NanoString, or microarray. The two PurIST subtypes, classical and basal-like, have meaningful associations with patient prognosis and treatment response. Within tumor cells, the basal-like subtype is defined by a poorer prognosis linked to early metastases and Folfirinox resistance, compared to the classical subtype, which is characterized by a progenitor epithelial phenotype. Within the stroma, the activated stroma is enriched in disorganized pro-tumor cancer associated fibroblasts with little extracellular matrix, while the inactive stroma is characterized by abundant and dense collagen secreted by more quiescent myofibroblasts. In addition, Puleo et al., Gastroenterology (2018), 155:1999-2013, described a PDA classification system based on gene expression analysis of formalin-fixed PDA samples. This classification system is based on molecular components extracted by an independent component analysis of transcriptomic data, and including notably 4 components that were named “classical”, “basal”, “stroma active” and “stroma inactive” based on correlation with biological signals. Both the PurIST subtypes of Rashid et al. and the molecular components of Puleo et al. are defined by gene expression derived, e.g., through RNA profiling. These approaches are limited by the quantity and quality of the samples (formalin fixation and low cellularity) as well as by the analytical delay that may restrict its application in routine care. In addition, tumors may harbor a mixture of several subtypes complicating their interpretation using bulk transcriptomic approaches and thereby limiting their clinical value. A recent study suggested that tumor cell architecture i.e. formation of glands, could partially predict tumor cell transcriptomic subtypes in primary resected tumors. This approach, while very interesting, requires highly trained pathologists and the analysis of the whole tumor.

### SUMMARY OF THE DESCRIPTION

Methods, systems, and devices for classification of an image are described herein, together with uses thereof.

In one aspect, disclosed herein is a computer-implemented method for processing a digital image of a pancreatic ductal adenocarcinoma (PDA) sample, the method comprising receiving a digital image of a PDA sample derived from a subject, applying a machine learning model to the digital image, and determining a PDA subtype for the image using the machine learning model: the machine learning model having been trained by processing a plurality of training images to predict PDA subtype, wherein the training images comprise a global label indicative of a known PDA subtype.

In some embodiments, the digital image is a H&E stained slide of a PDA sample.

In some embodiments, the known PDA subtype is assigned based on gene expression profiling (e.g., RNAseq or Nanostring) of a PDA sample derived from the same source as the training image. In some embodiments, the known PDA subtype is classified according to the PurIST classification scheme. In some embodiments, the known PDA subtype is classical and/or basal-like. In some embodiments, the known PDA subtype is classified according to the molecular subtype classification scheme. In some embodiments, the known PDA subtype is classified according to the molecular component profiling scheme. In some embodiments, the known PDA subtype is Classic, Basal, StromaActiv, or StromaInactive. In other embodiments, the known PDA subtype comprises a continuous score assigned to each training image which corresponds to one or both of the following classifications: classical and basal-like. In other embodiments, the known PDA subtype comprises a continuous score assigned to each training image which corresponds to one or more, two or more, three or more, or four of the following classifications: Classic, Basal, StromaActiv, or StromaInactive.

In some embodiments, the step of determining a PDA subtype for the image comprises determining a continuous score representing the likelihood that the tissue represented in the image belongs to one of two PDA subtypes. For example, the model can generate a score for the image (or for individual tiles derived from the image) between a first value and a second value, where a score closer to the first value indicates a higher likelihood that the tissue represented in the image belongs to a first subtype, and a score closer to the second value indicates a higher likelihood that the tissue represented in the image belongs to a second subtype. In some embodiments described herein, the model generates a score for the image between 0) and 1. As the score approaches 0), the model is predicting a higher likelihood that the tissue represented in the image is of a first subtype (e.g., Classical), and as the score approaches 1, the model is predicting a higher likelihood that the tissue represented in the image is of a second subtype (e.g., Basal). In this example, an image assigned a score of 0.9 is more likely to contain tissue of the Basal subtype than an image assigned a score of 0.7. Similarly, an image assigned a score of 0.2 is more likely to contain tissue of the Classic subtype than an image assigned a score of 0.4. An image assigned a score of 0.5 has approximately equal likelihood of containing tissue of the Classic subtype and the Basal subtype.

In other embodiments, the step of determining a PDA subtype for the image comprises determining one or more scores representing one or more PDA subtype features of the tissue sample represented in the image. For example, the model can assign a value to the image (or to individual tiles derived from the image) that quantifies how strongly particular subtype features are represented in the image (or in individual tiles derived therefrom). In this way, a molecular profile for the PDA sample can be determined, by compiling scores assigned to various PDA subtype features within the image. For example, in some embodiments, the model can determine a score for each of the following PDA subtype features within the image (or a tile derived therefrom): Classic, Basal, StromaActiv, StromaInactive. Accordingly, in this embodiment, the model will generate four scores, each indicating the degree to which the tissue sample represented in the image (or tile) contains Classic features, Basal features, StromaActiv features, or StromaInactive features. For example, the model can assign each image, or tile derived therefrom, a Classic features score between 0-1, with a score near 0 indicating that the tissue sample represented in the image contains very few Classic features, and a score near 1 indicating that the tissue sample represented in the image contains many Classic features. In addition, the model can assign the image, or tile derived therefrom, a Basal features score between 0-1, a StromaActiv features score between 0-1, and a StromaInactive features score between 0-1. In this way, a molecular profile for the sample can be determined, by assessing the scores and determining that the PDA sample has, for example, a high representation of Basal and StromaActiv features, and a minimal representation of Classic and StromaInactive features. The foregoing description presents a general framework for a model provided herein. The particular PDA subtype features determined by the model, and the range of possible scores to be assigned to each feature, can be adjusted by, for example, the labels assigned to the training images.

The foregoing method can also include one or more additional image processing or preprocessing steps as provided herein, including but not limited to (i) selecting one or more tumoral tissue segments present in the image: (ii) tiling the tumoral tissue segments into a set of tiles: and/or (iii) performing feature extraction on the set of tiles to extract a set of features.

In some aspects, provided herein is computer-implemented method for processing a digital image of a pancreatic ductal adenocarcinoma (PAC) sample, the method comprising receiving a digital image of a PAC sample derived from a subject, applying a machine learning model to the digital image, and determining a PAC classification for the image using the machine learning model, wherein the machine learning model has been trained by processing a plurality of training images.

In some embodiments, the digital image is a whole slide image (WSI).

In some embodiments, the method further comprises one or more image pre-processing steps.

For example, in some embodiments, the image pre-processing steps comprise one or more (i.e., one, two, or three) of the following: a. removing background segments from the image: b. tiling the digital image into a set of tiles: and c. performing a feature extraction on the set of tiles, or a subset thereof, to extract a set of features from the set of tiles. In some embodiments, the image pre-processing steps comprise (a), (b), and (c).

In some embodiments, the PAC classification is made at the slide level. In other embodiments, the PAC classification is made at the tile level.

In some embodiments, the PAC classification classifies a tile as containing neoplastic regions, or not containing neoplastic regions, wherein the neoplastic regions can comprise tumor cells and/or tumor-associated stromal cells. In some embodiments, the PAC classification is a continuous score representing the likelihood that the PAC sample represented in the tile contains neoplastic regions.

In some embodiments, the PAC classification classifies a tile as containing tumor cells, or containing stromal regions. In some embodiments, the PAC classification is a continuous score representing the likelihood that the PAC sample represented in the tile contains tumor cells, or contains stromal regions.

In some embodiments, the PAC classification comprises a continuous score representing the likelihood that the PAC sample represented in the image belongs to one of two PDA subtypes.

In some embodiments, the continuous score represents the likelihood that the PAC sample represented in the image as belongs to a PurIST PAC subtype selected from Classical or Basal-like.

In some embodiments, the PAC classification comprises one or more continuous scores representing the prevalence of one or more PDA subtype features in the tissue sample represented in the tile.

In some embodiments, the PAC classification comprises one or more continuous scores which reflects the degree to which a tile belongs to one or more molecular PAC subtypes selected from Classic, Basal, StromaActiv, and StromaInactive.

In some embodiments, the PAC classification comprises four continuous scores, each representing the prevalence of a PDA subtype feature selected from Classic, Basal, StromaActiv, and StromaInactive in the tissue sample represented in the tile.

In an exemplary embodiment, the present disclosure relates to a computer-implemented method of determining the pancreatic ductal adenocarcinoma (PDA) subtype corresponding to a PDA classification scheme of a subject having PDA. The method can include receiving a digital image of a histologic section of a PDA sample derived from the subject, and preprocessing the image to select one or more tumoral tissue segments present in the image. The tumoral tissue segments can include epithelial tumor cells and stroma regions. The method can also include tiling the tumoral tissue segments into a set of tiles, and performing a feature extraction on the set of tiles to extract a set of features from the set of tiles. The method can also include determining a PDA subtype for each of the tumoral tissue segments from the set of features using a machine learning model. The machine learning model can be trained for the PDA classification scheme, and each of the PDA subtypes for the one or more tumoral tissue segments can be a PDA subtype of the PDA classification scheme. In some embodiments, the method can also include computing one or more PDA molecular component scores for each tile of the set of tiles using the machine learning model. The machine learning model can be further trained to compute a score for each PDA molecular component including: Classic, Basal, StromaActiv, StromaInactive.

In some embodiments, the PDA classification scheme is one of a number of PDA classification schemes. In some embodiments, each of the PDA classification schemes includes a number of possible PDA subtypes.

In some embodiments, the histologic section of the PDA sample has been stained with a dye. In some embodiments, the dye is Haemotoxylin and Eosin (H&E).

In some embodiments, the digital image is a whole slide image (WSI).

In some embodiments, the PDA sample is a primary pancreatic ductal adenocarcinoma, or a portion thereof. In some embodiments, the PDA sample is a metastatic pancreatic ductal adenocarcinoma, or a portion thereof. In some embodiments, the metastatic pancreatic ductal adenocarcinoma, or portion thereof, is derived from the liver of the subject.

In some embodiments, the preprocessing step includes: (i) removing background segments from the image, and/or (ii) removing non-tumoral tissue segments from the image. In some embodiments, removing background segments from the image is performed using a convolutional neural network. In some embodiments, removing non-tumoral tissue segments from the image is performed by a model trained to distinguish neoplastic from normal regions in PDA. In some embodiments, the preprocessing step includes (i) and (ii).

In some embodiments, feature extraction is performed using Momentum Contrast or Momentum Contrast v2, as taught in Dehaene et al., Self-Supervision Closes the Gap Between Weak and Strong Supervision in Histology, Dec. 7, 2020.

In some embodiments, the PDA classification scheme is one of PurIST classification scheme and Molecular Component profiling scheme. The PurIST classification can include classical (also referred to as “classic”) and basal-like (also referred to as “basal”) subtypes. The Molecular Component profiling scheme can include: Classic, Basal, StromaActiv, and StromaInactive components. In some embodiments, the PDA classification scheme assigns one of the foregoing classifications to the PDA image. In some embodiments, the PDA classification scheme assigns a continuous score representing each subtype and/or each component to the PDA image. In other embodiments, the PDA classification scheme assigns a continuous score representing each subtype and/or each component to each tile within the PDA image.

In some embodiments, determining the PDA subtype for each of the tumoral tissue segments includes performing an analysis of the set of features extracted from the set of tiles using the machine learning model to generate a subtype score corresponding to each tile in the set of tiles. In some embodiments, determining the PDA subtype includes computing a PurIST score at a slide level based on an analysis of the set of features extracted from the set of tiles. In some embodiments, the machine learning model has been trained using a number of training images that include digital images of histologic sections of a PDA samples derived from subjects of known PDA subtype of the PDA classification scheme. In some embodiments, the training images each include a global label indicative of the known PDA subtype.

In some embodiments, the PDA classification scheme is PurIST and the global label is one of a Classical and Basal-like PurIST PDA subtype. In some embodiments, the machine learning model is a Deep Multiple Instance Learning model. In some embodiments, the PDA profiling scheme is Molecular Component and the global label is a value for each of Classic, Basal, StromaActiv, StromaInactive molecular components. In some embodiments, the machine learning model is a Weldon model.

In some embodiments, the known PDA subtype is identified using a gene expression profile of the PDA sample. In some embodiments, the gene expression profile includes RNAseq data or NanoString data. In some embodiments, the method can also include pooling the component scores corresponding to each tile in a plurality of tiles, to generate a component score corresponding to the digital image, where the component score corresponding to the digital image is indicative of a molecular component with highest predicted score.

In some embodiments, the method also includes overlaying the digital image with information representative of the component score of each tile in the set of tiles, to generate a digital image labeled with information representative of the component score of each tile in the set of tiles. In some embodiments, the information representative of the component score of each tile includes a label indicative of a molecular component with highest predicted score of the one or more tumoral tissue segments contained in the tile.

In some embodiments, the method also includes selecting the PDA classification scheme(s).

Also provided herein is a digital image of a histologic section of a PDA sample, wherein tumoral tissue segments within the image comprise labels associating the one or more tumoral tissue segments with one or more of a plurality of PDA subtypes, wherein the digital image is generated in accordance with a computer-implemented method set forth herein.

In some embodiments, the method also includes analyzing all PDA molecular component scores corresponding to a single tumor of a patient: determining a proportion of slides of the single tumor corresponding to different PDA molecular component scores: and generating a tumor-level PDA molecular component score based on the proportion of slides of the single tumor corresponding to different PDA molecular component scores.

In some aspects, provided herein is a machine readable medium having executable instructions to cause one or more processing units to perform a method for processing a digital image of a pancreatic ductal adenocarcinoma (PAC) sample, the method comprising receiving a digital image of a PAC sample derived from a subject, applying a machine learning model to the digital image, and determining a PAC classification for the image using the machine learning model, wherein the machine learning model has been trained by processing a plurality of training images.

In some embodiments, the digital image is a whole slide image (WSI).

In some embodiments, the method further comprises one or more image pre-processing steps.

For example, in some embodiments, the image pre-processing steps comprise one or more (i.e., one, two, or three) of the following: a. removing background segments from the image: b. tiling the digital image into a set of tiles: and c. performing a feature extraction on the set of tiles, or a subset thereof, to extract a set of features from the set of tiles. In some embodiments, the image pre-processing steps comprise (a), (b), and (c).

In some embodiments, the PAC classification is made at the slide level. In other embodiments, the PAC classification is made at the tile level.

In some embodiments, the PAC classification classifies a tile as containing neoplastic regions, or not containing neoplastic regions, wherein the neoplastic regions can comprise tumor cells and/or tumor-associated stromal cells. In some embodiments, the PAC classification is a continuous score representing the likelihood that the PAC sample represented in the tile contains neoplastic regions.

In some embodiments, the PAC classification classifies a tile as containing tumor cells, or containing stromal regions. In some embodiments, the PAC classification is a continuous score representing the likelihood that the PAC sample represented in the tile contains tumor cells, or contains stromal regions.

In some embodiments, the PAC classification comprises a continuous score representing the likelihood that the PAC sample represented in the image belongs to one of two PDA subtypes.

In some embodiments, the continuous score represents the likelihood that the PAC sample represented in the image as belongs to a PurIST PAC subtype selected from Classical or Basal-like.

In some embodiments, the PAC classification comprises one or more continuous scores representing the prevalence of one or more PDA subtype features in the tissue sample represented in the tile.

In some embodiments, the PAC classification comprises one or more continuous scores which reflects the degree to which a tile belongs to one or more molecular PAC subtypes selected from Classic, Basal, StromaActiv, and StromaInactive.

In some embodiments, the PAC classification comprises four continuous scores, each representing the prevalence of a PDA subtype feature selected from Classic, Basal, StromaActiv, and StromaInactive in the tissue sample represented in the tile.

In some aspects, provided herein is a machine readable medium having executable instructions to cause one or more processing units to perform a method for determining the pancreatic ductal adenocarcinoma (PDA) subtype corresponding to a PDA classification scheme of a subject having PDA, the method comprising: receiving a digital image of a histologic section of a PDA sample derived from the subject: preprocessing the image to extract a set of features, wherein, the preprocessing includes, tiling the digital image into a set of tiles, and performing a feature extraction on the set of tiles to extract a set of features from the set of tiles: selecting a subset of tiles that represent one or more tumoral tissue segments, wherein the subset of tiles includes a subset of features and the one or more tumoral tissue segments can comprise epithelial tumor cells and stroma regions: and determining a PDA subtype for the digital image from at least the subset of features using a machine learning model, wherein the machine learning model is trained for the PDA classification scheme and each of the PDA subtypes for the one or more tumoral tissue segments is a PDA subtype of the PDA classification scheme.

### BRIEF DESCRIPTION OF THE DRAWINGS

The present invention is illustrated by way of example and not limitation in the figures of the accompanying drawings in which like references indicate similar elements.

FIG. 1 illustrates an example flow diagram for a method of applying a trained machine learning model to predict a PDA molecular subtype score, according to embodiment of the present disclosure.

FIG. 2 illustrates an example flow diagram for a method of training and validating a DL model, according to embodiments of the present disclosure.

FIG. 3 illustrates an example flow diagram for a method of determining the pancreatic ductal adenocarcinoma (PDA) subtype corresponding to a PDA classification scheme of a subject having PDA, according to embodiments of the present disclosure.

FIG. 4 illustrates example flow diagrams for methods of generating a tumor-level PDA subtype score, according to embodiments of the present disclosure.

FIGS. 5A-5C are graphs showing validation results of the trained DL model, according to some embodiments of the present disclosure.

FIG. 6 is a graph showing overall survival for univariate/binary in the BJN cohort, according to embodiments of the present disclosure.

FIG. 7 is a graph showing overall survival for multivariate in the TCGA-PAAD cohort, according to embodiments of the present invention.

FIG. 8A illustrates an example set of Basal tiles, according to some embodiments of the present disclosure.

FIG. 8B illustrates an example set of Classic tiles, according to some embodiments of the present disclosure.

FIG. 9 illustrates an example of a computer system, which may be used in conjuncture with the embodiments described herein.

FIG. 10 presents a schematic of the workflow described in detail in Example 1.

FIG. 11A-11C provide flow charts illustrating the study design described in Example 1. FIG. 11A provides a description of the cohorts. The discovery cohort was composed of 202 patients (surgical specimens) from 3 centers. A tissue carrot (diameter 600 μm) was taken from a block for RNA profiling. HES slides (at least 2/tumor) were digitized for PACpAInt analysis. In most cases the tissue carrot and the HES did not come from the same block. The workflow was similar in the first validation cohort BJN_Unmatched (surgical specimens). For the 2 next validation cohorts (BJN-Matched (surgical specimens) and EUS_FNB (liver metastases, fine needle biopsies)), the same block was used for RNA extraction after microdissection for neoplastic area selection and to generate the HES slide that was digitized and analyzed with PACpAInt. In addition, in the BJN-Matched cohort, all the remaining tumor slides were also digitized for PACpAInt analysis. Finally, in the TCGA_PAAD validation cohort (surgical specimens), in contrast to all the other cohorts, the RNA was extracted from frozen material, not formalin-fixed paraffin-embedded. Similarly to the discovery cohort, the tissue analyzed by RNAseq was not spatially matched with the digitized slides. FIG. 11B provides a flow chart of the slide-level prediction. At the whole slide level (global classification of the whole slide), the multistep PACpAInt model first recognizes neoplastic areas (PACpAInt-Neo module), then assesses the basal-like of classical status (PACpAInt-B/C module) or the molecular components (PACpAInt-Comp module). FIG. 11C provides a flow chart of the tile-level prediction. In this setting, all the tiles (small square, 112 um wide) were analyzed and reported individually. The multistep PACpAInt model first recognizes neoplastic tile (PACpAInt-Neo module), then recognize tumor cell and stroma (PACpAInt-Cell type module) then assesses the molecular components (PACpAInt-Comp module) allowing the deep study of intra-tumor heterogeneity.

FIG. 12A-12C describe the identification of neoplastic areas by PACpAInt-Neo. FIG. 12A graphically depicts the performance of PACpAInt to identify neoplastic area in the BJN and TCGA_PAAD validation cohorts. FIG. 12B provides images of 2 example cases of neoplastic areas identified with H&E (left), PACpAInt-Neo segmentation (right) and zooms (center) of neoplastic (red/upper top and upper bottom panels) and non-neoplastic (green/lower top and lower bottom panels) areas. FIG. 12C presents representative tiles identified as neoplastic and non-neoplastic by PACpAInt-Neo in the TCGA_PAAD validation cohort.

FIG. 13A presents representative tiles identified as classical or basal-like by PACpAInt in the validation BJN cohort. FIG. 13B graphically depicts the performance of PACpAInt to identify molecular subtypes in validation cohort “BJN unmatched” (surgical specimens), i.e. where the slides analyzed and the tissue used for RNAseq are not spatially matched.

FIG. 14A presents representative tiles identified as classical or basal-like by PACpAInt-B/C in the TCGA_PAAD validation cohort. FIG. 14B graphically depicts the performance of PACpAInt-B/C to identify molecular subtypes at the whole slide level area in the TCGA_PAAD validation cohort.

FIG. 15 graphically depicts the performance of PACpAInt to identify molecular subtypes in the validation cohort “BJN matched” (surgical specimens), i.e., where the slides analyzed and the tissue used for RNAseq are spatially matched.

FIG. 16 graphically depicts the performance of PACpAInt to identify molecular subtypes in liver fine needle biopsies (FNB).

FIG. 17A presents the results of multivariate analyses of clinical/pathological factors and PACpAInt, demonstrating an independent prognostic value of the later on overall survival. FIG. 17B presents the results of multivariate analyses of clinical/pathological factors and PACpAInt-B/C on disease free survival in the BJN validation cohort. ***:p<0.001; **:p<0.01; *:p<0.05; +:p<0.1; −:p>0.1.

FIG. 18A presents the results of multivariate analyses of RNA-defined molecular subtype (PurIST-RNA) on overall survival (BJN validation cohort). FIG. 18B presents the results of multivariate analyses of RNA-defined molecular subtype (PurIST-RNA) on disease free survival (BJN validation cohort). ***:p<0.001; **:p<0.01; *:p<0.05; +:p<0.1; −:p>0.1.

FIG. 19 describes the application of PACpAInt to all the tumor slides (n=660) of 77 cases defined as classic by RNAseq on a single sampling. Top panel: The PACpAInt score estimating the “basalness” of each slide is represented on the Y axis while patients (1 to 77) are lined along the X axis. Each spot represents a slide. Cases with all their slides showing a low PACpAInt score (<0.2) were called “pure” classic compared to more heterogeneous tumors called “mixed” classic. Bottom panel: Kaplan Meyer analysis of overall survival comparing “pure” and “mixed” classical tumors. **:p<0.01.

FIG. 20A presents the performance of PACpAInt to identify tumor and stroma cells in the BJN (top) and TCGA_PAAD (bottom) validation cohorts. FIG. 20B presents representative tiles identified as tumor cells or stroma by PACpAInt in the TCGA_PAAD validation cohort. FIG. 20C presents the correlation between the tumor cell/stroma ratio computed by PACpAInt (y-axis) and with pan-cytokeratin immunohistochemistry (x-axis)

FIG. 21 presents multivariate analyses of clinical/pathological factors and PACpAInt-cell type computed tumor/stroma ratio on disease free (left) and overall (right) survival in the BJN validation cohort. ***:p<0.001; **:p<0.01; *:p<0.05; +:p<0.1; −:p>0.1.

FIG. 22 presents the correlation at the slide level between the tumor and stromal components defined by RNAseq or PACpAInt on the BJN unmatched (left panel) or matched (right panel) validation cohorts.

FIG. 23 presents the PACpAInt tumor and stroma score in tiles identified as classical, basal like, stroma active or inactive (analysis on 100K tiles).

FIG. 24 presents the correlation between slide-wise median stromal and epithelial scores by PACpAInt-Comp.

FIG. 25 presents a new classification into four subtypes, based on the tile scores of PACpAInt for the Classical and Basal components: main classical (classical), intermediary, hybrid, and main basal (basal). For each column-wise patient is first shown the 99th percentile basal-like and classical scores and second the proportion of tumor tiles for different levels of basal-like and classical differentiation.

FIG. 26 presents a Kaplan Meyer analysis of overall survival comparing main classical, intermediary, hybrid and main basal-like tumor (left panel), and a Kaplan Meyer analysis of disease-free survival comparing main classical, intermediary, hybrid and main basal-like tumor (right panel).

FIG. 27 presents a Kaplan Meyer analysis of overall survival (left panel) and disease-free survival (right panel) comparing tumors with less than 5%, 5 to 20%, and more than 20% of their tumor tiles being identified as basal-like.

FIG. 28 presents multivariate analyses of clinical/pathological factors, and PACpAInt-Comp computed amount of basal-like tile, on overall (top) and disease free (bottom) survival.

### DETAILED DESCRIPTION

A method and apparatus of a device that identifies pancreatic ductal adenocarcinoma (abbreviated interchangeably as “PAC” or “PDA”) features, e.g., subtypes, is described. In the following description, numerous specific details are set forth to provide thorough explanation of embodiments of the present invention. It will be apparent, however, to one skilled in the art, that embodiments of the present invention may be practiced without these specific details. In other instances, well-known components, structures, and techniques have not been shown in detail in order not to obscure the understanding of this description.

Reference in the specification to “one embodiment” or “some embodiments” means that a particular feature, structure, or characteristic described in connection with the embodiment can be included in at least one embodiment of the invention. The appearances of the phrase “in one embodiment” in various places in the specification do not necessarily all refer to the same embodiment. The term “exemplary” is used herein in the sense of “example,” rather than “ideal.” From this disclosure, it should be understood that the invention is not limited to the examples described herein.

For any methods described herein, the ordering of steps as presented, whether in the text or in an accompanying flow diagram, should not be taken to mean that those steps must be performed in the order presented, unless otherwise specified or required by context. Rather, the order of steps presents one embodiment of the methods provided, and in general such steps may alternatively be performed in a different order or simultaneously. The processes depicted in the figures that follow, are performed by processing logic that comprises hardware (e.g., circuitry, dedicated logic, etc.), software (such as is run on a general-purpose computer system or a dedicated machine), or a combination of both. Although the processes are described below in terms of some sequential operations, it should be appreciated that some of the operations described may be performed in different order. Moreover, some operations may be performed in parallel rather than sequentially.

Histology is the field of study relating to the microscopic features of biological specimens. Histopathology refers to the microscopic examination of specimens, e.g., tissues, obtained or otherwise derived from a subject, e.g., a patient, in order to assess a disease state. Histopathology specimens generally result from processing the specimen, e.g., tissue, in a manner that affixes the specimen, or a portion thereof, to a microscope slide. For example, thin sections of a tissue specimen may be obtained using a microtome or other suitable device, and the thin sections can be affixed to a slide. To assist in the visualization of the specimen, the specimen may optionally be further processed, for example, by applying a stain. Many stains for visualizing cells and tissues have been developed. These include, without limitation, Haemotoxylin and Eosin (H&E), methylene blue, Masson's trichome, Congo red, Oil Red O, and safranin. H&E is routinely used by pathologists to aid in visualizing cells within a tissue specimen. Hematoxylin stains the nuclei of cells blue, and eosin stains the cytoplasm and extracellular matrix pink. A pathologist visually inspecting an H&E stained slide can use this information to assess the morphological features of the tissue. However, H&E stained slides generally contain insufficient information to assess the presence or absence of particular biomarkers by visual inspection. Visualization of specific biomarkers (e.g., protein or RNA biomarkers) can be achieved with additional staining techniques which depend on the use of labeled detection reagents that specifically bind to a marker of interest, e.g., immunofluorescence, immunohistochemistry, in situ hybridization, etc. Such techniques are useful for determining the expression of individual genes or proteins, but are not practical for assessing complex expression patters involving a large number of biomarkers. Global expression profiling can be achieved by way of genomic and proteomic methods using separate samples derived from the same tissue source as the specimen used for histopathological analysis. Notwithstanding, such methods are costly and time consuming, requiring the use of specialized equipment and reagents, and do not provide any information correlating biomarker expression to particular regions within the tissue specimen, e.g., particular regions within the H&E stained image.

Pancreatic ductal adenocarcinoma (“PAC”, “PDA”, or “PDAC”) has a high level of molecular heterogeneity. Genomic tools such as RNAseq and Nanostring have been used to identify and classify clinically relevant PDA subtypes (see, e.g., Rashid et al., Clinical Cancer Research (2020): 26:82-92 and Puleo et al., Gastroenterology (2018), 155:1999-2013).

Digital images of histology slides, e.g., H&E stained slides, allow computational assessment of tissue specimens, in addition to or alternatively to visual inspection by a pathologist. Provided herein are computer-implemented methods, and associated systems and computer-readable media, for determining PDA subtype based on a digital image of a PDA tissue section, without the need for genomic or proteomic analysis.

Computing methods used for implementing the methods provided herein can include, for example, machine learning, artificial intelligence (AI), deep learning (DL), neural networks, classification and/or clustering algorithms, and regression algorithms.

A used herein, the term “digital image” refers to an electronic image represented by a collection of pixels which can be viewed, processed and/or analyzed by a computer. In some embodiments, a digital image can be acquired by means of a digital camera or other optical device capable of capturing digital images from a slide, or portion thereof. In other embodiments, a digital image can be acquired by means of scanning a non-electronic image of a slide, or portion thereof. In some embodiments, the digital image used in the applications provided herein is a whole slide image. As used herein, the term “whole slide image (WSI),” refers to an image that includes all or nearly all portions of a tissue section, e.g., a tissue section present on a histology slide. In some embodiments, a WSI includes an image of an entire slide. In other embodiments, the digital image used in the applications provided herein is a selected portion of a tissue section, e.g., a tissue section present on a histology slide. In some embodiments, a digital image is acquired after a tissue section has been treated with a stain, e.g., H&E.

As used herein, the “region of interest” of an image could be any region semantically relevant for the task to be performed, in particular, regions corresponding to tissues, organs, bones, cells, body fluids, etc. when in the context of histopathology.

As used herein, “PDA classification scheme” refers to a classification framework for determining one or more PDA subtype(s). Exemplary PDA classification schemes provided herein include PurIST classification, and molecular component classification. According to the PurIST classification scheme, a PurIST subtype score can be generated (e.g., between 0 and 1), which represents the likelihood that the PDA sample represented in a digital image is of the Basal or Classic subtype. In some embodiments, a PurIST subtype score is determined at the slide level, where a single score is assigned to the digital image. According to the molecular component classification scheme, a molecular component subtype score can include a vector corresponding to the values for each molecular component selected from: Classic, Basal, StromaActiv, and StromaInactive. In some embodiments, a molecular component subtype score is determined at the tile level, where a molecular subtype score is assigned to individual tiles derived from the digital image.

As used herein, classifying an image describes associating to a particular image a label from a predetermined list of labels. In the context of histopathology, the classification could be a diagnosis classification. In one embodiment, the classification can be binary, e.g. the labels are simply “healthy”/“not healthy,” or “Basal-like”/“Classical.” In other embodiments, there could be more than two labels, for example labels corresponding to different diseases, labels corresponding to different stages of a disease, labels corresponding to different kinds of diseased tissue, etc. For example, in some embodiments there are more than two labels indicative of PDA subtype. In some embodiments, the labels can include the molecular component classifications Classic, Basal, StromaActiv, and StromaInactive.

A “PDA subtype,” or “PAC subtype” describes a subgroup of pancreatic ductal adenocarcinoma sharing certain common features. For example, the PurIST PAC subtypes “Classic” or “Classical” and “Basal” or “Basal-like” were initially defined by commonalities in gene expression shared by cancers of the same subtype, and distinct from cancers of the other subtype, as described by Rashid et al., Clinical Cancer Research (2020); 26:82-92. These commonalities and differences in gene expression can be determined, for example, by RNA expression profiling, e.g., RNAseq. In addition, as described herein, the cancers of each subtype surprisingly share common morphological features that can be identified using deep learning models. Accordingly, the models provided herein can determine whether a subject's pancreatic ductal adenocarcinoma is of the “Classic” or “Basal” subtype by analyzing an image of the PDA (e.g., a digital image of an H&E stained histological section derived from the PDA) as described herein, without the need to perform RNA expression profiling on a sample of PDA tissue.

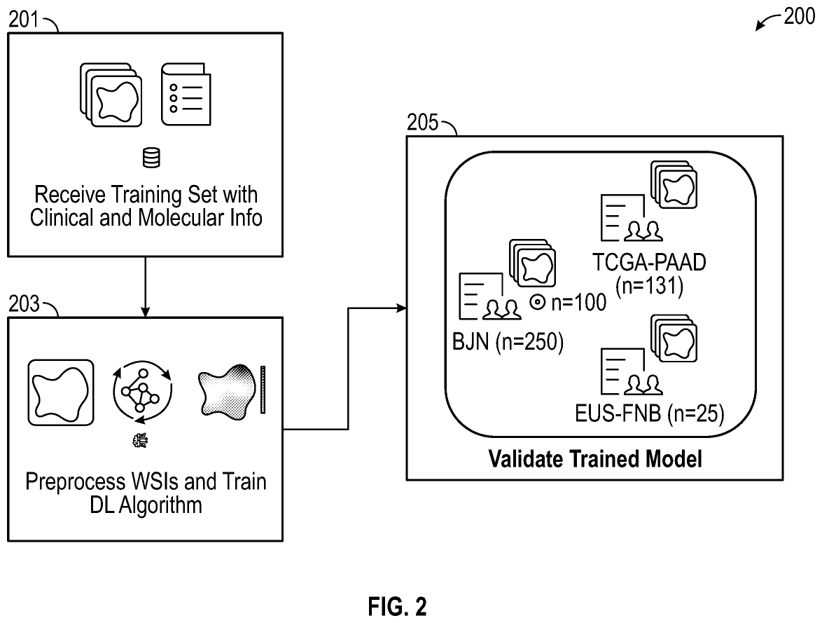

The deep learning methods described herein also allow the identification and characterization of additional PAC subtype features based on the morphology of the PAC sample. As described in Example 1 below; in one embodiment employing the methods set forth herein, four PAC subtype features were identified, and given the classifications “Classic”, “Basal”, “StromaActiv”, and “StromaInactive,” based on the PDA transcriptomic components described by Puleo et al., Gastroenterology (2018), 155:1999-2013 (see Puleo et al., FIG. 2: the transcriptomic component described by Puleo as “basal-like tumor” was assigned the designation “Basal” in the study described herein: the transcriptomic component described by Puleo as “classical tumor” was assigned the designation “Classic” in the study described herein: the transcriptomic component described by Puleo as “activated stromal” was assigned the designation “StromaActiv” in the study described herein: and the transcriptomic component described by Puleo as “inflammatory stromal” was assigned the designation “StromaInactive” in the study described herein).

As used herein, predicting a global score of an image describes calculating a value representative of a characteristic of the image as a whole. For example, a slide-level score is a score that is applied to entire image (e.g., a whole slide image). This is in contrast with a tile-level score, which is applied to individual tiles derived from the image following a tiling process, as described herein. In the context of the systems and methods provided herein, a global score could be a score indicative of PDA subtype. In other embodiments, the global score can include a risk score correlated with prognosis (i.e. a survival rate, a survival expectancy, etc.), a risk score correlated with response to a treatment (i.e. the probability of a treatment to be effective, a variation of expectancy, etc.), or any significant parameter for diagnosis.

The terms “server,” “client,” and “device” are intended to refer generally to data processing systems rather than specifically to a particular form factor for the server, client, and/or device.

A multistep approach is provided that uses deep learning (DL) models to predict tumor components and their molecular subtypes on routine histological preparations. In one particular embodiment, 424 WSIs corresponding to 202 resected PAC from three centers with clinical and transcriptomic data were assembled and used as a discovery set (i.e. training set). An independent cohort of 250 cases was used as a validation set, as well as PAC from TCGA (n=134), and an independent cohort of 25 liver biopsies. Tumor regions from slides of the discovery set were annotated to train a multistep DL model that first recognizes tumor tissue, and then predicts molecular subtypes.

The techniques disclosed herein demonstrate the value of histology-based DL models for complex tumor transcriptomic subtyping in PAC. The DL models can predict the neoplastic areas (i.e. tumor areas) of a whole slide image, determine molecular PAC subtypes at the whole slide level on routine histological preparations, and distinguish the tumor cells/stroma compartments and predict their respective molecular subtype at the tile level to decipher the intratumor heterogeneity on a massive scale. The present disclosure provides the first AI-based PAC subtyping tool, finally opening the possibility of patient molecular stratification in routine care and clinical trials. Additional benefits include assessing intra-tumor heterogeneity using an external cohort of cases with slides of a tumor, and validating models in a cohort from a prospective clinical trial. A further benefit of the present disclosure is the ability to identify the location of different molecular components within a WSI.

Accordingly, provided herein is a computer-implemented method for processing a digital image of a pancreatic ductal adenocarcinoma (PDA) sample. The method can comprise (i) receiving a digital image of a PDA sample derived from a subject, (ii) applying a machine learning model to the digital image, and (iii) determining a PDA subtype for the image using the machine learning model, wherein the machine learning model is a model previously trained by processing a plurality of training images to predict PDA subtype. In some embodiments, the digital image is a whole slide image of a PDA tissue section. In some embodiments, the PDA tissue section has been stained with a stain, such as hematoxylin and eosin. In some embodiments, the plurality of training images comprises a plurality of whole slide images of training PDA tissue sections, wherein the training PDA tissue sections are derived from a tumor of known PDA subtype. In some embodiments, the plurality of training images are each stained with a stain, e.g., hematoxylin and eosin. In some embodiments, each of the plurality of training images comprises a global label indicative of a known PDA subtype. In some embodiments, the plurality of training images lack local annotations of PDA features. In some embodiments, the machine learning model provides a score representing the likelihood that the PDA sample derived from the subject has the predicted PDA subtype.

In some embodiments, the foregoing method can further comprise additional preprocessing steps to select one or more tumoral segments present in the digital image. In some embodiments, the method can further comprise tiling the image, or the tumoral segments within the image, into a set of tiles. In some embodiments, the method can further comprise performing a feature extraction on the set of tiles, to extract a set of features therefrom.

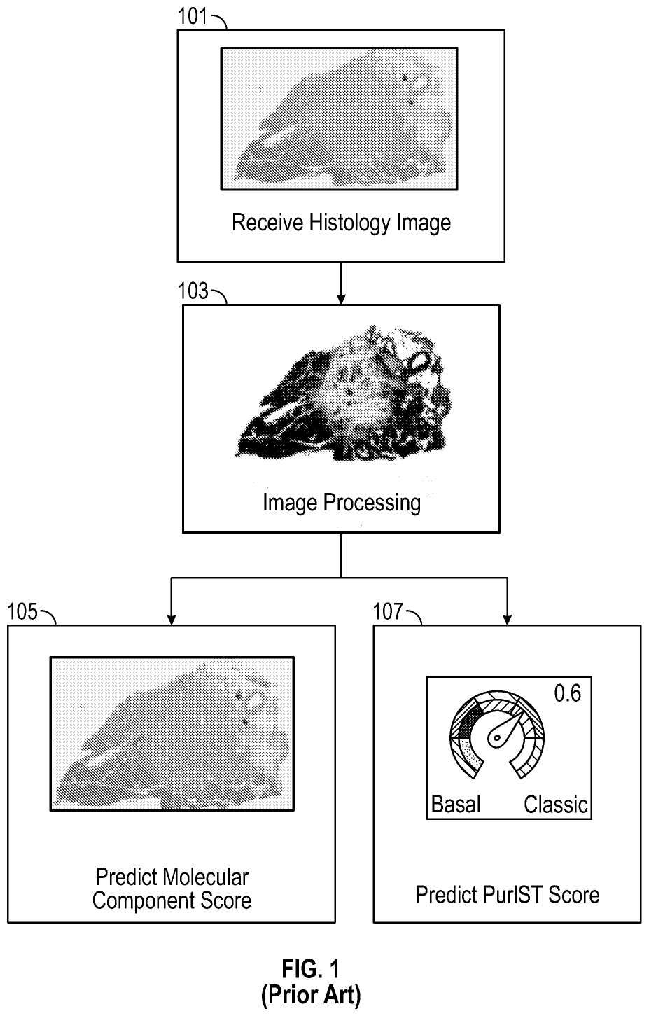

FIG. 1 illustrates an example flow diagram for a method of applying a trained machine learning model to predict a PDA molecular subtype score, according to embodiments of the present disclosure. At operation 101, a histology image is received. The histology image can include a digital WSI, in some embodiments.

In one embodiment, the input histology image is derived from a patient tissue sample that may be known or suspected to contain a PDA tumor. In some embodiments, the input image can comprise a tissue section that has been stained to visualize the underlying tissue structure, for example, with hematoxylin and eosin (H&E). Other common stains that can be used to visualize tissue structures in the input image include, for example, Masson's trichome stain, Periodic Acid Schiff stain, Prussian Blue stain, Gomori trichome stain, Alcian Blue stain, or Ziehl Neelsen stain.

At operation 103, image processing is performed. In some embodiments, image processing can include removing background portions of the image, tiling the image, feature extraction, and/or identifying tumor regions within an image. The application of DL algorithms to histological data is a challenging problem, particularly due to the high dimensionality of the data, and the small size of the available datasets. Therefore, a preprocessing pipeline composed of multiple steps can be used to reduce dimensionality and clean the data.

In some embodiments, preprocessing includes detecting the tissue regions of the image. Identification of the tissue regions within a WSI can be performed prior to or following additional image processing functions, such as tiling. In one embodiment, a neural network (e.g., U-Net) can be used to segment parts of the image that contain matter, and discard artifacts such as blur, pen marks, etc. as well as the background portion of the image where no tissue is present.

Tiling the image, (or the image minus the background) can include dividing the original image (or the image minus the background), into smaller images that are easier to manage, called tiles. In one embodiment, the tiling operation is performed by applying a fixed grid to the whole-slide image, using a segmentation mask generated by a segmentation method, and selecting the tiles that contain tissue, or any other region of interest, for the later classification process. In order to reduce the number of tiles to process even further, additional or alternative selection methods can be used, such as random subsampling to keep only a given number of slides.

In some embodiments, the original image can be down-sampled in order to make the image segmentation step less computationally expensive. In some embodiments, some of the image analysis is performed at a tile level (which is a subsection of the image), using the semantic segmentation on a down-sampled version of the image does not degrade the quality of the segmentation. This allows the use of down-sampled image without degrading the quality of the segmentation. Then, to obtain the segmentation mask for the original full resolution image, one simply needs to upscale the segmentation mask generated by the neural network.

For example, and in one embodiment, an image (or the image minus the background) can be divided into tiles of fixed size (e.g., each tile having a size of 224×224 pixels). Alternatively, the tile size can be smaller or larger. In this example, the number of tiles generated depends on the size of the matter detected, and can vary from a few hundred tiles to 50,000 or more tiles. In one embodiment, the number of tiles is limited to a fixed number that can be set based on at least the computation time and memory requirements (e.g., 10,000 tiles). In some embodiments, at least 5%, 10%, 15%, 20%, 30% or more of a tile must have been detected as foreground by the U-Net model discussed above for it to be considered as a tile of matter. Once the WSI has been divided into tiles, the image processing can extract features from each tile.

In some embodiments, the feature extraction is performed using a self-supervised model. For example, the self-supervised model MoCo v2 can be used to extract features from the tiles. In some embodiments, feature extraction can be performed generally in accordance with the methods described by Dehaene et al., “Self-Supervision Closes the Gap Between Weak and Strong Supervision in Histology” (2020), arXiv (https://arxiv.org/pdf/2012.03583). In some embodiments, about 1,000 to 5,000, about 1,000 to 3,000, or about 2,000 relevant features can be extracted from each tile. In some embodiments, 2,048 features are extracted from each tile, such that at the end of the preprocessing pipeline, a slide is represented by a matrix of size (nₜᵢₗₑₛ, 2048).

Once the features have been extracted from the tiles, a trained DL model can be used to predict the neoplastic areas of a tile. In one embodiment, the tumor detection model can be trained at the tile level based on tumor annotations provided by an expert pathologist. The TurnNet model (also referred to as PACpAInt-Neo) described herein includes a multi-layer perceptron with a single layer of 128 hidden neurons, followed by ReLU activation. In some embodiments, the tumor detection model classifies a tile by assigning the tile a score that represents the likelihood that the tile contains neoplastic regions. Such neoplastic regions can contain tumor cells, and/or tumor-associated stromal cells. In some embodiments, the tile score can be a value between 0 and 1, where one end point (e.g., 0) indicates a very high likelihood that the tile does not contain neoplastic regions, and the other end point (e.g., 1) indicates a very high likelihood that the tile does contain neoplastic regions. In some embodiments, a tile may be classified as a tumor tile if it has a tumor prediction score larger than a threshold value, for example, 0.5. Tumoral tissue segments can include, in some embodiments, epithelial tumor cells and stromal regions.

At operation 105, the image (or the image tiles) can be analyzed by trained DL models in order to predict a PDA subtype. PuriNet (also referred to as PACpAInt-B/C) and CompoNet (also referred to as PACpAInt-Comp) are two DL models that were trained on a discovery cohort (training set) to predict, respectively, a PurIST classification (Classic or Basal) and continuous sample weights of molecular components (Classic, Basal, StromaActiv, and StromaInactive). In some embodiments, both PuriNet and CompoNet can use the same image (e.g., WSI) preprocessing pipeline described above, including identifying the tissue regions, tiling the image, and feature extraction. In some embodiments, the model can be trained using a training set of images that contain one or more global labels indicative of PDA subtype. In some embodiments, the PDA molecular subtype labels of training set images are based on a gene expression profile (e.g., generated using RNA profiling) or a protein expression profile of a PDA sample derived from the same subject as that of the training image. By way of example, in some embodiments, the PDA subtype labels associated with each image in the training set can be based on an evaluation of the gene expression profile of a paired sample from the same subject that has been classified as classical or basal-like according to the PurIST criteria set forth in Rashid et al., Clinical Cancer Research (2020)): 26:82-92. In some embodiments, the PDA molecular subtype labels associated with each image in the training set can be based on an evaluation of the gene expression profile of a paired sample from the same subject that has been classified as Classic, Basal, StromaActiv, or StromaInactive based on the PDA transcriptomic components described by Puleo et al., Gastroenterology (2018), 155:1999-2013 (see Puleo et al., FIG. 2). In some embodiments, the PDA molecular subtype labels associated with each image in the training set can be based on an evaluation of the gene expression profile of a paired sample from the same subject that has been classified as pure classical, immune classical, pure basal-like, stroma activated, or desmoplastic according to the criteria also set forth in Puleo et al.

FIG. 2 illustrates an example flow diagram for a method 200 of training and validating a DL model, according to embodiments of the present disclosure. In order to validate locally the patterns predictive of Classic and Basal identified by the CompoNet model, GATA6 and VIM IHCs were performed on a number of slides. The tile scores for Basal and Classic components in Classic and Basal regions were then compared according to the IHCs.

At operation 201, the method may begin by receiving a training set with clinical and molecular information. For example, the training set can include a cohort of H&E WSIs with annotations.

At operation 203, the WSIs can be preprocessed, and the DL model can be trained to predict molecular information from the preprocessed images. In some embodiments, the model can be trained using training images that include digital images of histologic sections of PDA samples derived from subjects of known PDA subtype. For example, each training image can include a global label indicative of a known PDA subtype.

At operation 205, the trained model can be validated. In this particular example, the trained model is validated using three validation cohorts: 1) Beaujon/BJN (n=150+n=100 perfect match RNAseq), 2) TCGA-PAAD (n=134), and 3) Beaujon Biopsies/EUS-FNB (n=25).

The area under the receiver operating characteristic curve (AUC) was used to quantify the capability of the model to distinguish Classic from Basal tumors, as assessed by the PurIST method. The same metric was also used to assess the performance of the tumor detection model to distinguish normal from tumoral regions. Delong's approach was used to compute confidence intervals at 95% confidence level. Pearson correlation was used to assess the performance of the CompoNet model to predict the molecular components. Survival analyses was used with univariate and multivariate Cox proportional hazards models implemented in the lifelines package of Python. Log-rank tests were used to compare survival distributions between population subgroups. The tests were two-tailed, and P values<0.05 were considered statistically significant. Clinical variables considered for multivariate analysis were common variables known to be associated with PAC prognosis, including for example: pN stage, differentiation, perinervous invasion, resection status, tumor size, vascular invasion, and/or adjuvant treatment yes/no.

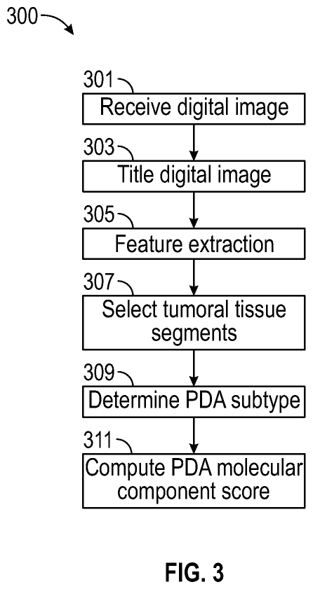

FIG. 3 illustrates an example flow diagram for a method 300 of determining the pancreatic ductal adenocarcinoma (PDA) subtype corresponding to a PDA classification scheme of a subject having PDA, according to embodiments of the present disclosure.

At operation 301, a digital image of a histologic section of a PDA sample derived from the subject is received. As discussed above, the digital image may be a WSI of a PDA tissue section. The PDA tissue section may have been stained with a stain, such as hematoxylin and eosin.

At operation 303, the image is tiled in order to break the WSI into smaller images that are easier to manage. In one embodiment, the tiling operation is performed by applying a fixed grid to the whole-slide image, using a segmentation mask generated by a segmentation method, and selecting the tiles that contain tissue, or any other region of interest for the later classification process. In order to reduce the number of tiles to process even further, additional or alternative selection methods can be used, such as random subsampling to keep only a given number of slides.

Once the WSI has been divided into tiles, the method 300 may continue at operation 305 with extracting features from each tile. In some embodiments, feature extraction is performed using a self-supervised model. For example, the self-supervised model MoCo v2 can be used to extract features from the tiles. In one embodiment, features may be extracted by applying a trained feature extractor that was trained with a contrastive loss DL algorithm using a training set of images. In one embodiment, the training set of images can include a set of annotated images that have PDA molecular subtype labels generated using RNA profiling.

Once the features have been extracted from the tiles, the method 300 may continue at operation 307 with selecting tumoral tissue segments using a trained DL model. In one embodiment, a tumor detection model, such as TurnNet, can be trained at the tile level based on tumor annotations provided by an expert pathologist. The TurnNet model includes a multi-layer perceptron with a single layer of 128 hidden neurons, followed by ReLU activation. In some embodiments, the tumor detection model classifies a tile by assigning the tile a score that represents the likelihood that the tile contains neoplastic regions. Such neoplastic regions can contain tumor cells, and/or tumor-associated stromal cells. In some embodiments, a tile may be classified as a tumor tile if it has a tumor prediction score larger than, for example, 0.5. In some embodiments, the tumoral tissue segments can include epithelial tumor cells and stroma regions.

At operation 309, a DL model is used to determine a PDA subtype by applying the model to tiles comprising one or more tumoral tissue segments identified at operation 307. In some embodiments, the determination of PDA subtype can be made at the tile level, while in other embodiments the determination of PDA subtype can be made at the slide level. PuriNet and CompoNet, which are also discussed above in reference to FIG. 1, are two DL models that were trained on a training set to predict, respectively, a PurIST classification (Classic or Basal) and continuous sample weights of molecular components (Classic, Basal, StromaActiv, and StromaInactive). In one embodiment, the machine learning model is trained for the PDA classification scheme, and each of the PDA subtypes to be assigned to the image, or to tiles derived from the image, is a PDA subtype of the PDA classification scheme. In some embodiments, the PDA classification scheme is PurIST, and the PDA subtypes include classic and/or basal. In other embodiments, the PDA classification scheme is molecular component, and the PDA subtypes include Classic, Basal, StromaActiv, and StromaInactive.

The PuriNet model can be used to obtain a score at the slide level, which represents the probability that the tissue represented in the image of the slide is Basal or Classic. In one embodiment, PuriNet was trained with the binary cross entropy as loss function. In some embodiments, the score is between 0 and 1, with a score at one end of the range (e.g., 0) representing a very high likelihood that the tissue contained on the slide is of the Classic subtype, and a score at the other end of the range (e.g., 1) representing a very high likelihood that the tissue contained on the slide is of the Basal subtype.

CompoNet, which was inspired by the WELDON algorithm, can be used to compute a set of one-dimensional embeddings for the tile features using a multi-layer perceptron (MLP). For each channel output of the MLP, the R=100 top and bottom scores can be averaged so that the model's output is a vector corresponding to the values of each molecular component: Classic, Basal, StromaActiv, and StromaInactive.

At optional operation 311, one or more PDA molecular component scores are computed for each tile of the set of tiles using the machine learning model. The machine learning model can also be trained to compute a score for each PDA molecular component including: Classic, Basal, StromaActiv, StromaInactive.

In some embodiments, the DL models can predict a PurIST classification and a molecular component sample weight at the tile level based on the features extracted from the tiles. Based on this tile-level knowledge, a PurIST score and a molecular component score can also be generated at the slide level.

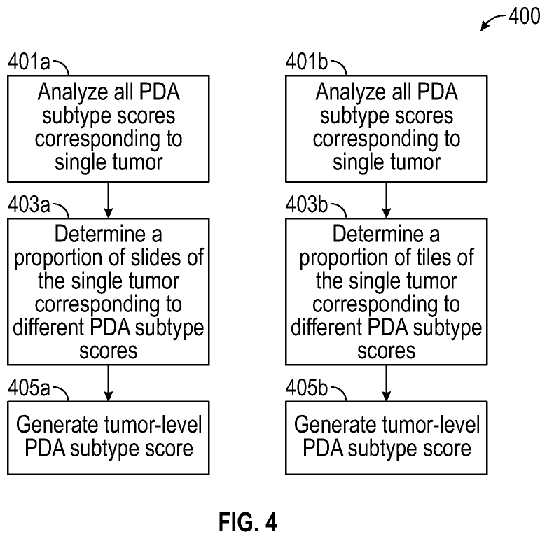

FIG. 4 illustrates an example flow diagram for a method 400 of generating a tumor-level PDA subtype score, according to embodiments of the present disclosure. According to one embodiment, the techniques described herein can be applied to multiple images of the same tumor. For example, a tumor biopsy can be performed, and multiple images can be generated of various sections of the tumor. These images can be analyzed in order to determine a PDA subtype score and provide a more complete assessment at the tumor-level.

At operations 401a and 401b, a number of PDA subtype scores corresponding to a single tumor of a patient are analyzed. In one embodiment, many sections of a single tumor are imaged and analyzed, according to the techniques described herein, in order to generate PDA subtype scores for images derived from different sections of the same tumor.

At operation 403a, the proportion of slides of the single tumor corresponding to different PDA subtype scores is determined. For example, if 100 slides of a single tumor are analyzed, the method can determine which proportion of those slides correspond to a given PDA subtype. This aggregation can also be done at the tile level, as shown in operation 403b, by determining which proportion of tiles derived from one or more images correspond to a given subtype.

At operation 405, a tumor-level PDA subtype score is generated based on the aggregate proportion of slides (405a) or tiles (405b) of the single tumor corresponding to different PDA subtypes. In this way, a PDA subtype score can be generated at a tumor-level. For example, if after analyzing 100 slides from a single tumor, it is determined that 85% of the slides include majority Basal subtype, then a tumor-level label of Basal can be assigned. In another example, a tumor-level label of 85% Basal can be assigned to the tumor, with the remaining score assigned based on the proportion of other subtypes identified in the tumor (e.g., 85% basal/15% classic, or 85% basal/5% Classic/8% StromaActiv/2% StromaInactive). As will be apparent to a person skilled in the art, the aggregation of PDA subtype scores at the tumor-level can be achieved in a number of ways. For example, in embodiments, where a PDA subtype score is assigned to each image at the slide level, the number of slides of each subtype can be determined and used to assign a tumor-level subtype label. In another example, where a PDA subtype score is assigned to each image at the tile level, the number of tiles of each subtype can be determined and used to assign a slide-level label. The number of slides of each subtype can then be determined and used to assign a tumor-level subtype label. In another example, wherein a PDA subtype score is assigned to each image at the tile level, the number of tiles of each subtype identified in the tumor (e.g., across multiple slides) can be determined and used to assign a tumor-level subtype label.

In some embodiments, a tumor-level subtype score is determined for the subtypes Classic, Basal, or both Classic and Basal. In other embodiments, a tumor-level subtype score is determined for the subtypes Classic, Basal, StromaActiv, and/or StromaInactive.

The foregoing operations can be used to identify the various subtypes of PAC that exist in a population of subjects, and/or can be used to determine the particular PAC subtype of an individual patient, using histopathological images (e.g., H&E stained images).

For example, in some aspects, provided herein is computer-implemented method for processing a digital image of a pancreatic ductal adenocarcinoma (PAC) sample, the method comprising receiving a digital image of a PAC sample derived from a subject, applying a machine learning model to the digital image, and determining a PAC classification for the image using the machine learning model, wherein the machine learning model has been trained by processing a plurality of training images.

In some embodiments, the digital image can be a whole slide image (WSI), which encompasses all of the tissue on a histology slide. The tissue can, in some embodiments, be stained to visualize morphological features of the PAC sample. For example, the sample can be stained with H&E, and/or other suitable dyes, such as those described herein.

In some embodiments, the method further comprises one or more image pre-processing steps.

For example, in some embodiments, the image pre-processing steps comprise one or more (i.e., one, two, or three) of the following: a. removing background segments from the image: b. tiling the digital image into a set of tiles: and c. performing a feature extraction on the set of tiles, or a subset thereof, to extract a set of features from the set of tiles. In some embodiments, the image pre-processing steps comprise (a), (b), and (c).

In some embodiments, the PAC classification is made at the slide level. In other embodiments, the PAC classification is made at the tile level.

In one embodiment, provided herein is computer-implemented method for identifying the neoplastic regions within a digital image of a pancreatic ductal adenocarcinoma (PAC) sample, the method comprising:
receiving a digital image of a PAC sample derived from a subject,
pre-processing the image, wherein the pre-processing comprises removing background segments from the image, tiling the image into a set of tiles, and performing a feature extraction on the set of tiles, or a subset thereof, to extract a set of features from the set of tiles, and
applying a machine learning model to the digital image, wherein the machine learning model assigns a tile score to each tile in the set of tiles, or a subset thereof, said tile score representing the likelihood that the tile contains neoplastic regions.

In some embodiments, the machine learning model has been trained by processing a plurality of training images, wherein the training images comprise digital images of a plurality of PAC samples, and wherein the digital images contain local annotations defining the neoplastic regions. In some embodiments, the neoplastic regions contain tumor cells. In other embodiments, the neoplastic regions contain tumor cells and associated stromal cells.

Once tiles have been assigned a tile score, the tiles can, in some embodiments, be filtered to select tiles above or below a threshold value. In this manner, tiles containing neoplastic regions, and/or tiles containing non-neoplastic regions, can be selected for further analysis. In some embodiments, tiles having been assigned a tile score can be mapped back to, or superimposed upon, their position in the digital image. This allows the digital image to be labeled to indicate the portions containing neoplastic regions, and the portions not containing neoplastic regions.

In one embodiment, provided herein is computer-implemented method for identifying the subtype of a pancreatic ductal adenocarcinoma (PAC) sample derived from a subject known or suspected of having PAC, the method comprising:
receiving a digital image of the PAC sample,
pre-processing the image, wherein the pre-processing comprises removing background segments from the image, tiling the image into a set of tiles, and performing a feature extraction on the set of tiles, or a subset thereof, to extract a set of features from the set of tiles,
selecting a subset of tiles that contain neoplastic regions, and
applying a machine learning model to the subset of tiles that contain neoplastic regions, wherein the machine learning model assigns a slide score to the subset of tiles, said slide score representing the likelihood that the PAC sample belongs to a PAC subtype.

In some embodiments, the machine learning model has been trained by processing a plurality of training images, wherein the training images comprise digital images of a plurality of PAC samples of known subtype, wherein the training images comprise a global (slide-level) label indicative of the known subtype. In some embodiments, the training images lack local annotations.

In some embodiments, the foregoing method is a method of identifying the PurIST subtype of a PAC sample, wherein the PurIST subtype is selected from Classic and Basal. In some embodiments, the training images each comprise a global label, indicating the PurIST subtype (e.g., Classic or Basal) of the PAC sample of known subtype represented therein.

In one embodiment, provided herein is computer-implemented method for identifying the subtype of a pancreatic ductal adenocarcinoma (PAC) sample derived from a subject known or suspected of having PAC, the method comprising:
receiving a digital image of the PAC sample,
pre-processing the image, wherein the pre-processing comprises removing background segments from the image, tiling the image into a set of tiles, and performing a feature extraction on the set of tiles, or a subset thereof, to extract a set of features from the set of tiles,
selecting a subset of tiles that contain neoplastic regions, and
applying a machine learning model to the subset of tiles that contain neoplastic regions, wherein the machine learning model assigns a tile score to each tile in the set of tiles, or a subset thereof, said tile score representing the likelihood that the PAC tissue represented in the tile belongs to a PAC subtype. In some embodiments, the tile score is a plurality of scores, each representing the likelihood that the PAC tissue represented in the tile belongs to a PAC subtype.

In some embodiments, the machine learning model assigns one, two, three, four, five, six, seven, eight, nine, ten, or more tile score(s) to each tile in the set of tiles, or a subset thereof, each tile score representing the likelihood that the PAC tissue represented in the tile belongs to a PAC subtype.

In some embodiments, the machine learning model has been trained by processing a plurality of training images, wherein the training images comprise digital images of a plurality of PAC samples of known subtype, wherein the training images comprise a global (slide-level) label indicative of the known subtype. In some embodiments, the training images lack local annotations.

In some embodiments, the foregoing method is a method of identifying the PurIST subtype of a PAC sample, wherein the PurIST subtype is selected from Classic and Basal. In some embodiments, the training images each comprise a global label, indicating the PurIST subtype (e.g., Classic or Basal) of the PAC sample of known subtype represented therein. In some embodiments, the PAC subtype is selected from Classic, Basal, StromaActiv, and StromaInactive.

Once tiles have been assigned a tile score, the tiles can, in some embodiments, be filtered to select tiles above or below a threshold value. In this manner, tiles containing one or more region(s) representing a particular PAC subtype, can be selected for further analysis. In some embodiments, tiles having been assigned a tile score can be mapped back to, or superimposed upon, their position in the digital image. This allows the digital image to be labeled to indicate the portions containing specific PAC subtypes.

In some embodiments, the techniques described herein can be used to identify the predominant PAC subtype of a PAC sample derived from a patient known or suspected of having PAC.

In some embodiments, the techniques described herein can be used to assess the level of subtype heterogeneity of a PAC sample derived from a patient known or suspected of having PAC.

In some embodiments, the foregoing methods can be performed on multiple digital images derived from the same PAC sample. For example, the methods can be performed on a plurality of digital images derived from serial tissue sections of a PAC sample. In other embodiments, the methods can be performed on a plurality of digital images derived from tissue sections obtained from various regions of a PAC sample. Assessing PAC subtypes using multiple tissue sections allows the uniformity or heterogeneity of a PAC sample to be assessed in three dimensions.

In some embodiments, the foregoing techniques can be used to identify the PurIST subtype (Classic or Basal) of a subject known or suspected to have pancreatic ductal adenocarcinoma (PAC). In some embodiments, the foregoing techniques can be used to identify the molecular subtype (Classic, Basal, StromaActiv, and StromaInactive) of a subject known or suspected to have pancreatic ductal adenocarcinoma (PAC).

The PAC subtypes described herein have prognostic significance for overall survival duration, and disease-free survival duration, both alone and in combination with other clinical characteristics. Accordingly, in some embodiments, provided herein is a method of facilitating the prognosis of a subject having PAC, by determining the PAC subtype in accordance with the methods and/or systems described herein, and correlating the PAC subtype with the prognosis of the subject. For example, in some embodiments, provided herein is a method of predicting the survival duration of a subject having PAC, comprising determining the PAC subtype in according with the methods and/or systems described herein, and correlating the PAC subtype with the survival duration of the subject. In other embodiments, provided herein is a method of predicting the disease-free survival duration of a subject having PAC, comprising determining the PAC subtype in according with the methods and/or systems described herein, and correlating the PAC subtype with the disease-free survival duration of the subject. In other embodiments, provided herein is a method of predicting the survival duration of a subject having PAC, comprising determining the proportion of tiles from a digital image of a PAC sample having a particular PAC subtype (e.g., basal, classic) in according with the methods and/or systems described herein, and correlating the proportion of PAC tiles having a particular PAC subtype with the survival duration of the subject. In other embodiments, provided herein is a method of predicting the disease-free survival duration of a subject having PAC, comprising determining the proportion of tiles from a digital image of a PAC sample having a particular PAC subtype (e.g., basal, classic) in according with the methods and/or systems described herein, and correlating the proportion of PAC tiles having a particular PAC subtype with the disease-free survival duration of the subject.

A machine readable medium having executable instructions to cause one or more processing units to perform any of the foregoing computer-implemented methods is also provided herein.

Also provided are systems for processing a digital image of a PDA sample, the system comprising: at least one memory storing instructions, and at least one processor configured to execute instructions to perform operations necessary to execute the computer-implemented methods provided herein.

These example embodiments are provided to illustrate representative applications of the systems and methods provided herein. These embodiments are merely exemplary. Additional models and methodologies can be used in accordance with the disclosure provided herein to develop and train machine learning models and/or systems for the purpose of PDA diagnosis and classification based in histopathology slide images.

Exemplary methods and techniques used in the example embodiments were as follows:

Transcriptome profiling and molecular subtyping. For the discovery cohort, microarray was used to determine PurIST subtype, as well as the molecular components, while for the validation cohorts RNASeq 3′ was used.

Preprocessing of whole-slide images. The application of deep-learning algorithms to histological data is a challenging problem, particularly due to the high dimensionality of the data (up to 100,000×100,000 pixels for a single whole-slide image) and the small size of available datasets. Therefore a preprocessing pipeline composed of multiple steps was used to reduce dimensionality and clean the data. The first step comprised detecting the tissue on the WSI: a U-Net neural network was used to segment part of the image that contains matter, and discard artifacts such as blur, pen marker etc., as well as the background. A second step comprised tiling the slide into smaller images, called “tiles”, of 112×112 μm (224×224 pixels). At least 20% of the tile must have been detected as foreground by the U-Net model to be considered as a tile of matter. A maximum of 8000 such tiles were then uniformly sampled from each slide. A third step comprised extracting features from each tile: 2,048 relevant features were extracted using a self-supervised model, namely MoCo v2, following the approach of Dehaene et al., “Self-Supervision Closes the Gap Between Weak and Strong Supervision in Histology (2020), arXiv (https://arxiv.org/pdf/2012.03583), incorporated by reference herein in its entirety. At the end of this preprocessing pipeline, each slide was represented by a matrix of size (nₜᵢₗₑₛ, 2048).

Tumor model structure. A tumor detection ML model, ‘TurnNet’, was trained at the tile level based on tumor annotations of 745 samples provided by an expert pathologist, which corresponds to a total of 17,292,173 million tiles. WSI preprocessing techniques described above (“Preprocessing of whole-slide images”) were used to obtain 2,048 features for each tile. The model comprises a multi-layer perceptron with a single layer of 128 hidden neurons, followed by ReLU activation. The model outputs a tumor prediction score for each tile, which indicates the presence or absence of tumor regions. The tumor detection model is applied to the WSI to select those tiles within the digital image that contain tumor regions. Such tumor regions may include tumor cells as well as stromal components of the tumor.

Molecular model structures. Two deep learning models were trained on the discovery cohort to predict the PurIST classification (‘PuriNet’) or the molecular components Classic, Basal, StromaActiv, StromaInactive (CompoNet) of a WSI. The two models use the same WSI preprocessing pipeline described above in “Preprocessing whole-slide images.” The TurnNet model was further applied on the tile features in order to select only tumor tiles (i.e. with tumor prediction score larger than 0.5).

PuriNet uses a similar architecture to that proposed by Ilse et al., “Attention-based deep multiple instance learning.” International conference on machine learning. PMLR. 2018. A linear layer with 128 neurons is applied to the embedding followed by a Gated Attention layer with 128 hidden neurons. A MLP with 128 and 64 hidden neurons and ReLU activations was then applied to the results. A final Sigmoid activation was applied to the output to obtain a score between 0 and 1, which represents the probability of the slide to be Basal or Classic. PuriNet was trained with the binary cross entropy as loss function.

CompoNet uses a similar architecture as the WELDON algorithm (see, for example, Thibaut Durand, Nicolas Thome, Matthieu Cord. WELDON: Weakly Supervised Learning of Deep Convolutional Neural Networks. 29th IEEE Conference on Computer Vision and Pattern Recognition (CVPR 2016), June 2016. Las Vegas, NV. United States). A set of one dimensional embeddings is computed for the tile features using a multi-layer perceptron (MLP) with 128 hidden neurons followed by 4 neurons and ReLU activation. For each channel output of the MLP, R=100 top and bottom scores were selected and averaged, so that the model output is a vector of size 4, corresponding to the values of each molecular component. PuriNet was trained with the mean squared error as loss function.

Spatial Validation. To validate locally the patterns predictive of Classic and Basal identified by CompoNet, GATA6 and VIM IHCs were performed on slides of BJN_matched. The tile scores for Basal and Classic components were compared with Classical and Basal regions according to immunohistochemistry (IHC).

Performance assessment and statistical methods. The area under the receiver operating characteristic curve (AUC) was used to quantify the capability of the model to distinguish Classic from Basal tumors as assessed by the PurIST method. The same metric was also used to assess the performance of the Tumor detection model to distinguish normal from tumoral regions. Delong's approach was used to compute confidence intervals at 95% confidence level. Pearson correlation was used to assess the performance of the CompoNet model to predict the molecular components. Survival analyses were performed with uni- and multivariate Cox proportional hazards models implemented in the lifelines package of Python. Log-rank tests were used to compare survival distributions between population subgroups. All tests were two-tailed, and P values<0.05 were considered statistically significant.

Clinical variables used in the multivariate analysis. Clinical variables considered for multivariate analysis were common variables known to be associated with PDA prognosis: pN stage, differentiation, perinervous invasion, resection status, tumor size, vascular invasion, adjuvant treatment yes/no.

A multistep DL model was trained on a cohort of 424 digital H&E WSIs derived from 202 resected PDA specimens from 3 centers (mean number of slides/case=2), in which the neoplastic areas were annotated by two pathologists. At least two hematoxylin-eosin (H&E) slides from surgical specimens were available for each patient, corresponding to a total of 424 slides.

The trained model was then validated on three independent cohorts, including a cohort of biopsies. The characteristics of the training cohort (i.e. the training set), and the three validation cohorts, are presented below in Table 1.

TABLE 1

| Cohort | Validation? | # Slides | # Patients | Other |
|---|---|---|---|---|
| Training | No | 424 | 202 | Multi-centric, resected primary |
| | | | | tumor |
| BJN_250 | Yes | 505 | 250 | Resected primary tumor |
| TCGA-PAAD | Yes | 132 | 132 | Multi-centric, resected primary |
| | | | | tumor |
| EUS-FNB | Yes | 25 | 25 | Fine needle liver biopsies |
| | | | | (metastasis) |

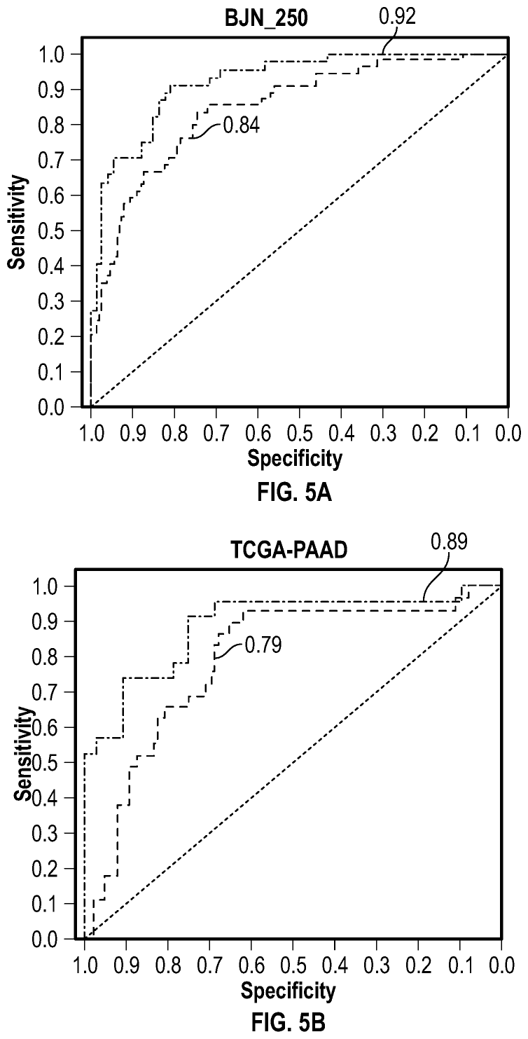

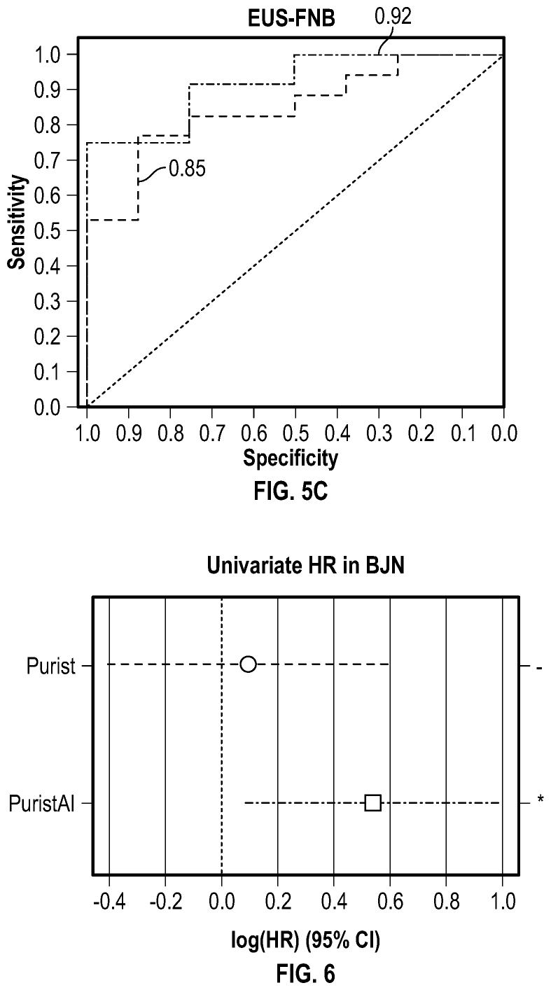

When applied to two distinct validation cohorts (BJN_250 and TCGA-PAAD), the model properly detected the neoplastic areas. Molecular transcriptomic subtype prediction was validated on two external cohorts of surgical specimens, BJN_250 (FIG. 5A) and TCGA-PAAD (FIG. 5B), with AUCs of 0.84 and 0.79, respectively. Interestingly, our model better predicted the subtypes when restricting to samples with a high-confidence RNA-defined molecular subtypes, and reached 0.92 AUC and 0.89 AUC in BJN_250 and TCGA_PAAD.

Because most PDA patients are diagnosed at the metastatic stage on liver biopsies, the model was tested on 25 fine needle liver biopsies with matched RNAseq data. The model performance was good (AUC=0.85 [0.69−1.0]) and like the surgical specimens, it improved in cases with a clear molecular subtype (AUC=0.92 [0.77−1.0] (FIG. 5C).

In one embodiment, molecular predication outputted by the trained DL model was independently associated in BJN_250 with overall survival (OS) and disease-free survival (DFS), in multivariate analysis.

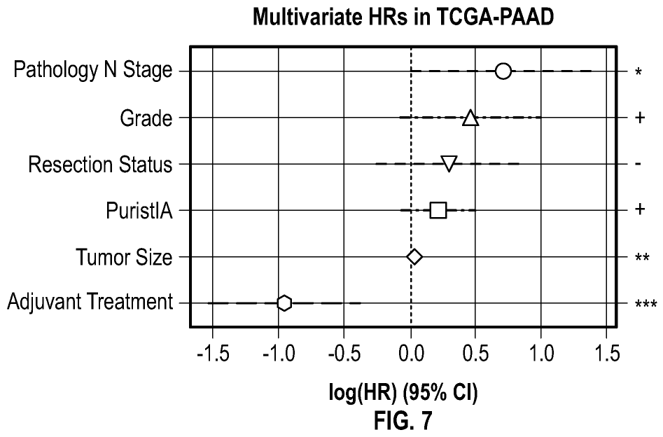

Graphs showing overall survival for univariate/binary for BJN (FIG. 6), and multivariate for TCGA-PAAD (FIG. 7), according to embodiments of the present disclosure.

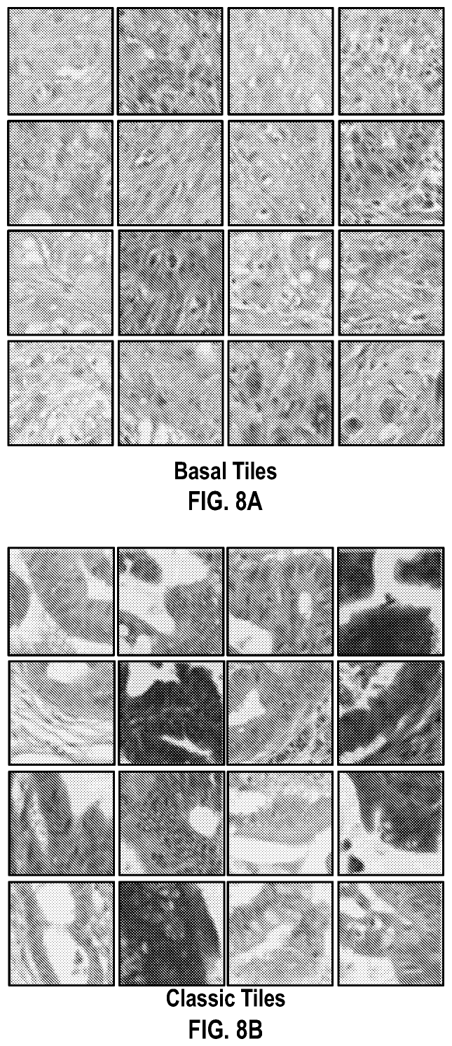

FIG. 8A illustrates an example set of Basal tiles, while FIG. 8B illustrates an example set of Classic tiles.

In another example embodiment, in the training set the Basal-like/Classical classification performance of the model by cross validation was 0.79 (AUC). The performance of the same model reached 0.86 AUC when restricted to samples with a high-confidence RNA-defined molecular subtype. Subtypes defined by the model were independently associated with overall survival in multivariate analysis (HR=2.56 [1.87−3.49], pval<0.001), and association was higher relatively to PurIST RNA subtypes (HR=1.60 [1.17−2.19] pval<0.001). In the validation cohort, the model had an overall AUC of 0.82, and 0.89 in the subset of “subtype-pure” tumors. In addition to demonstrating the value of histology-based deep learning models for tumor subtyping in PAC, these results also show the limit of molecular-based subtyping in highly heterogeneous samples.

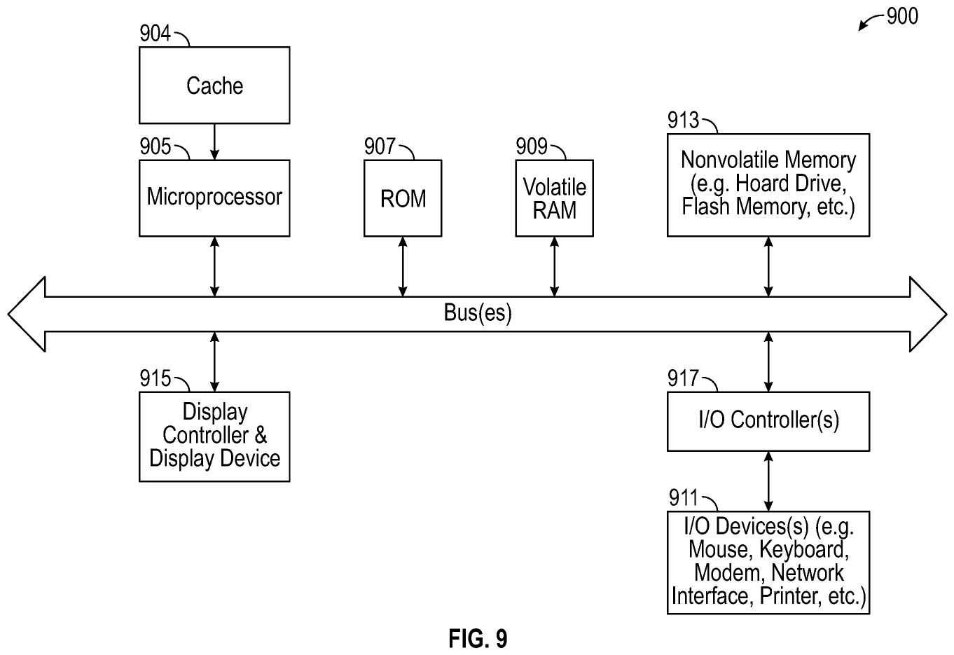

As shown in FIG. 9, the computer system 900, which is a form of a data processing system, includes a bus 903 which is coupled to a microprocessor(s) 905 and a ROM (Read Only Memory) 907 and volatile RAM 909 and a non-volatile memory 913. The microprocessor 905 may include one or more CPU(s), GPU(s), a specialized processor, and/or a combination thereof. The microprocessor 905 may be in communication with a cache 904, and may retrieve the instructions from the memories 907, 909, 913 and execute the instructions to perform operations described above. The bus 903 interconnects these various components together and also interconnects these components 905, 907, 909, and 913 to a display controller and display device 915 and to peripheral devices such as input/output (I/O) devices 911 which may be mice, keyboards, modems, network interfaces, printers and other devices which are well known in the art. Typically, the input/output devices 911 are coupled to the system through input/output controllers 917. The volatile RAM (Random Access Memory) 909 is typically implemented as dynamic RAM (DRAM), which requires power continually in order to refresh or maintain the data in the memory.

The nonvolatile memory 913 can be, for example, a magnetic hard drive or a magnetic optical drive or an optical drive or a DVD RAM or a flash memory or other types of memory systems, which maintain data (e.g. large amounts of data) even after power is removed from the system. Typically, the nonvolatile memory 913 will also be a random access memory although this is not required. While FIG. 9 shows that the nonvolatile memory 913 is a local device coupled directly to the rest of the components in the data processing system, it will be appreciated that the present invention may utilize a nonvolatile memory which is remote from the system, such as a network storage device which is coupled to the data processing system through a network interface such as a modem, an Ethernet interface or a wireless network. The bus 903 may include one or more buses connected to each other through various bridges, controllers and/or adapters as is well known in the art.

Portions of what was described above may be implemented with logic circuitry such as a dedicated logic circuit or with a microcontroller or other form of processing core that executes program code instructions. Thus processes taught by the discussion above may be performed with program code such as machine-executable instructions that cause a machine that executes these instructions to perform certain functions. In this context, a “machine” may be a machine that converts intermediate form (or “abstract”) instructions into processor specific instructions (e.g., an abstract execution environment such as a “virtual machine” (e.g., a Java Virtual Machine), an interpreter, a Common Language Runtime, a high-level language virtual machine, etc.), and/or, electronic circuitry disposed on a semiconductor chip (e.g., “logic circuitry” implemented with transistors) designed to execute instructions such as a general-purpose processor and/or a special-purpose processor. Processes taught by the discussion above may also be performed by (in the alternative to a machine or in combination with a machine) electronic circuitry designed to perform the processes (or a portion thereof) without the execution of program code.

The present invention also relates to an apparatus for performing the operations described herein. This apparatus may be specially constructed for the required purpose, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, and magnetic-optical disks, read-only memories (ROMs), RAMS, EPROMS, EEPROMs, magnetic or optical cards, or any type of media suitable for storing electronic instructions, and each coupled to a computer system bus.

A machine readable medium includes any mechanism for storing or transmitting information in a form readable by a machine (e.g., a computer). For example, a machine readable medium includes read only memory (“ROM”); random access memory (“RAM”): magnetic disk storage media: optical storage media: flash memory devices: etc.

An article of manufacture may be used to store program code. An article of manufacture that stores program code may be embodied as, but is not limited to, one or more memories (e.g., one or more flash memories, random access memories (static, dynamic or other)), optical disks, CD-ROMs, DVD ROMs, EPROMS, EEPROMs, magnetic or optical cards or other type of machine-readable media suitable for storing electronic instructions. Program code may also be downloaded from a remote computer (e.g., a server) to a requesting computer (e.g., a client) by way of data signals embodied in a propagation medium (e.g., via a communication link (e.g., a network connection)).

The preceding detailed descriptions are presented in terms of algorithms and symbolic representations of operations on data bits within a computer memory. These algorithmic descriptions and representations are the tools used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be kept in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “segmenting,” “tiling,” “receiving,” “computing,” “extracting,” “processing,” “applying,” “augmenting,” “normalizing,” “pre-training,” “sorting,” “selecting,” “aggregating,” “sorting,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The processes and displays presented herein are not inherently related to any particular computer or other apparatus. Various general-purpose systems may be used with programs in accordance with the teachings herein, or it may prove convenient to construct a more specialized apparatus to perform the operations described. The required structure for a variety of these systems will be evident from the description below. In addition, the present invention is not described with reference to any particular programming language. It will be appreciated that a variety of programming languages may be used to implement the teachings of the invention as described herein.

### Example 1: Deep Learning Approach to Identify Molecular Subtypes of Pancreatic Adenocarcinoma on Histology Slides

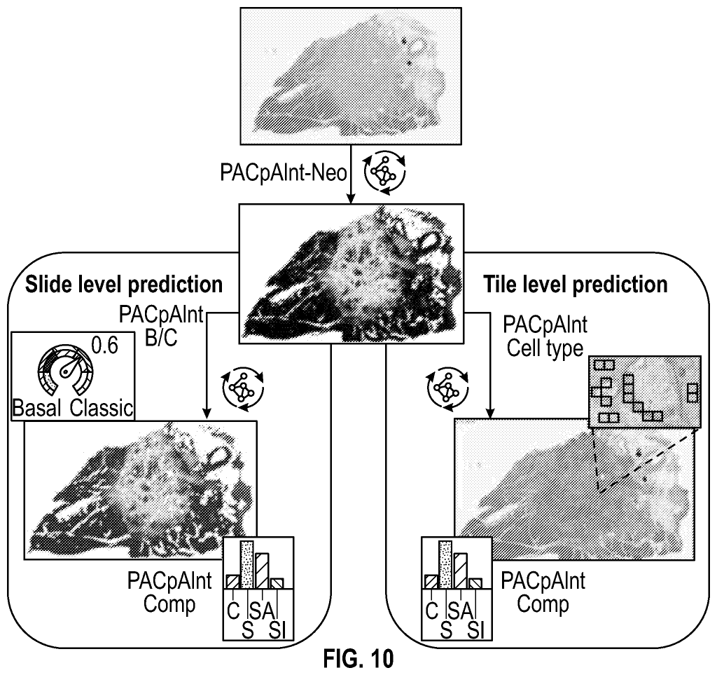

Described herein is a multistep approach that uses deep learning models to determine tumor cell type and molecular phenotype on routine histological preparations at a resolution that allows one to decipher intratumor heterogeneity on a massive scale (FIG. 10). The model was trained and validated on multicentric cohorts using 1796 slides (602 patients) with matched transcriptome.

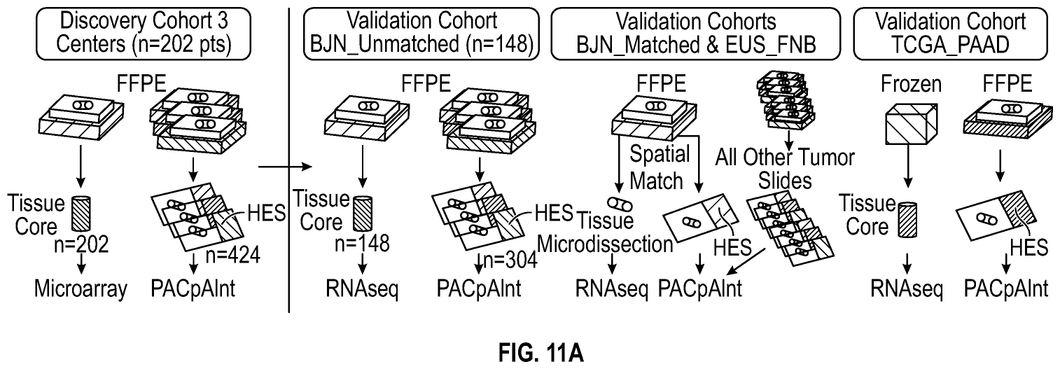

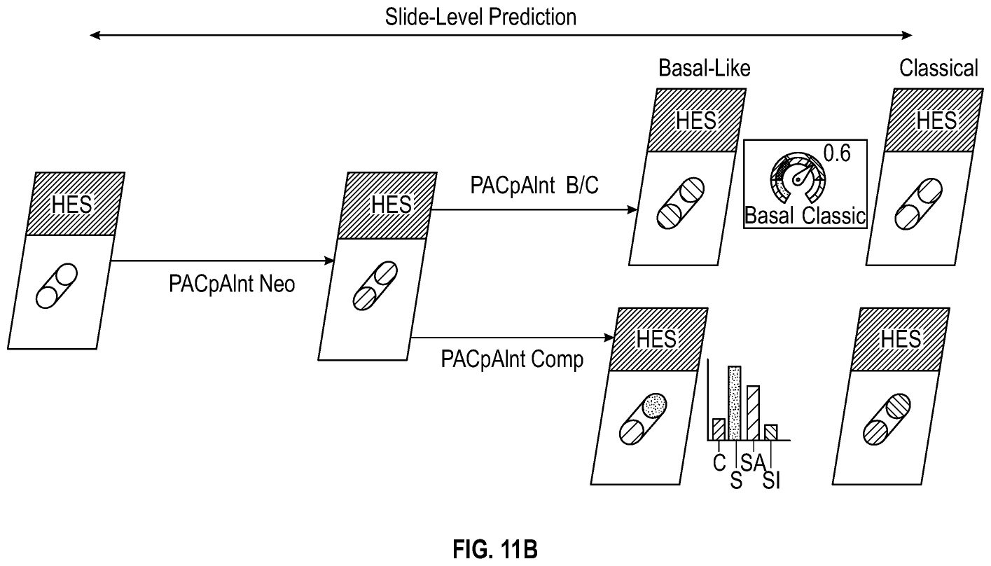

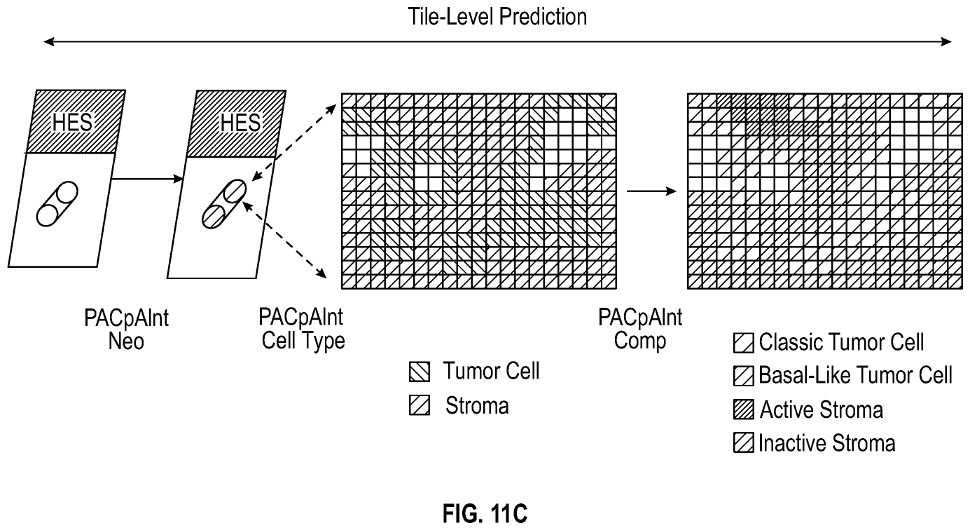

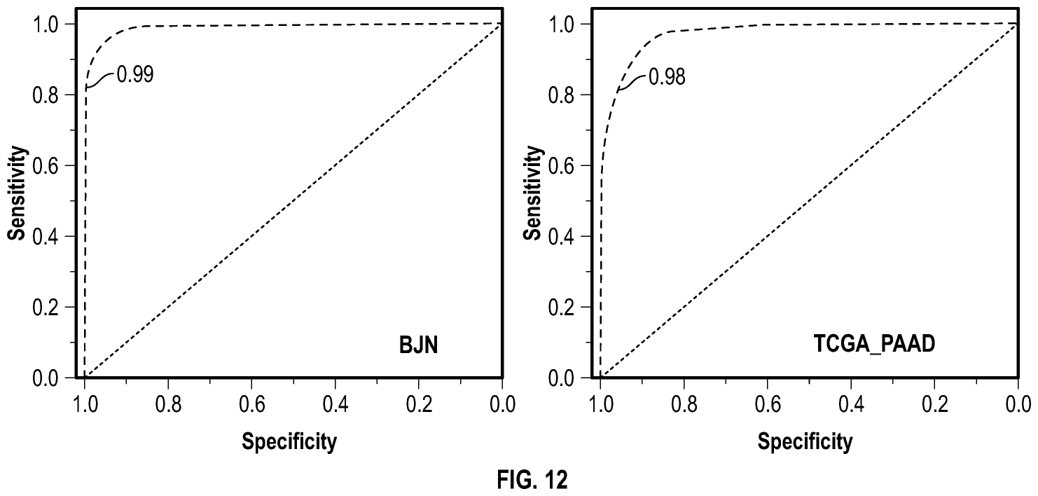

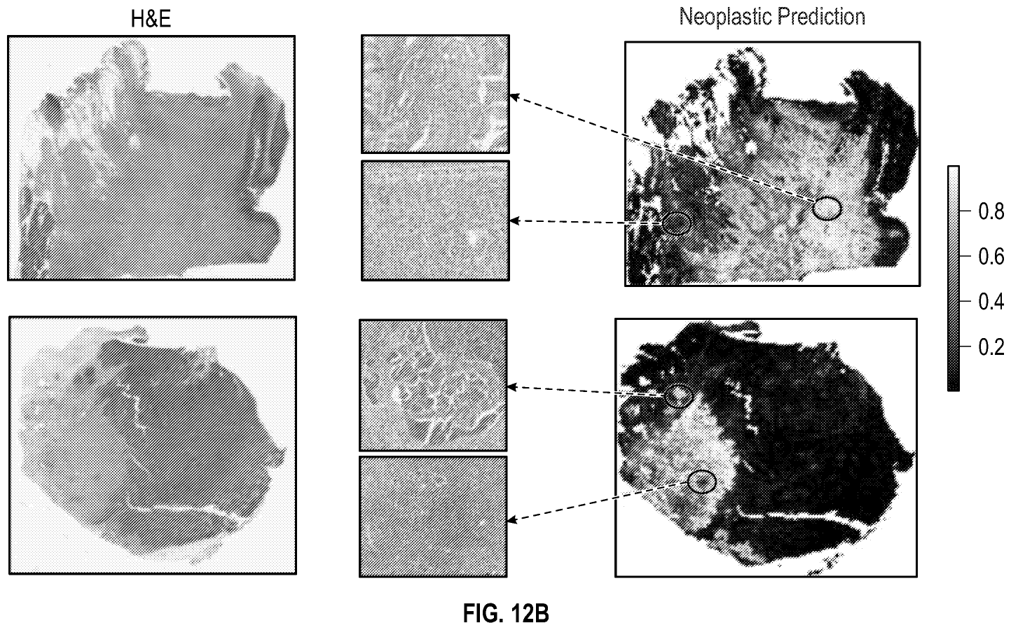

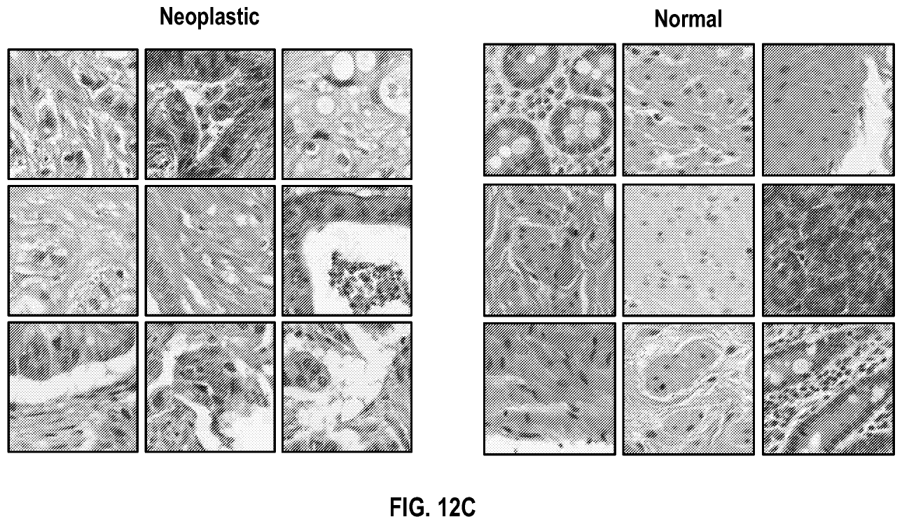

Deep learning models were trained on a discovery cohort composed of 424 whole-slide histological images from 202 resected PAC from 3 centers (mean number of slides/case=2) in which the neoplastic areas were annotated by two pathologists (FIG. 11A). A first model was developed to predict neoplastic areas (PACpAInt-Neo) and when applied to two distinct validation cohorts, the model properly detected the neoplastic areas with an AUC of 0.99 and 0.98 respectively (FIG. 12A).

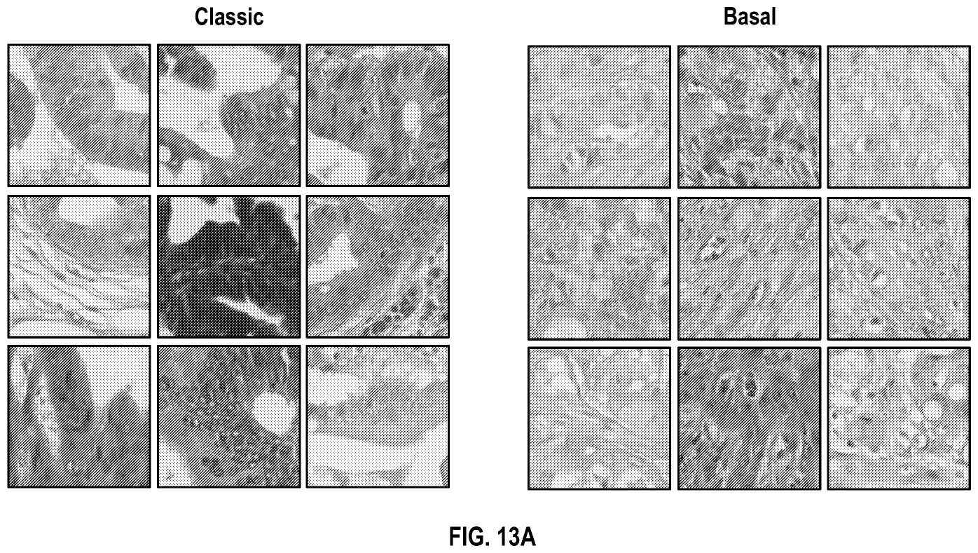

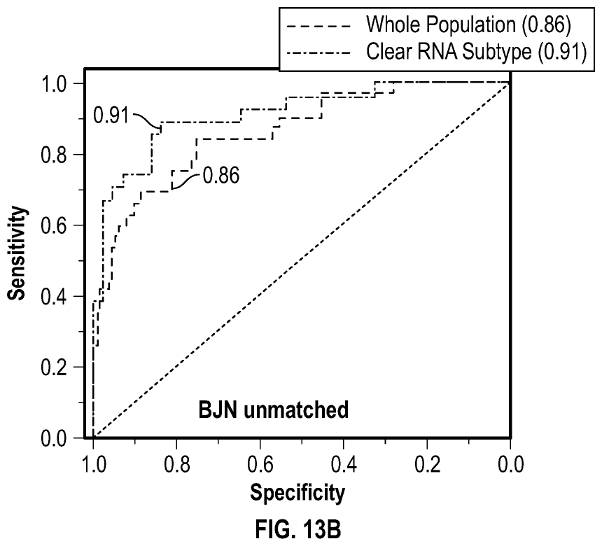

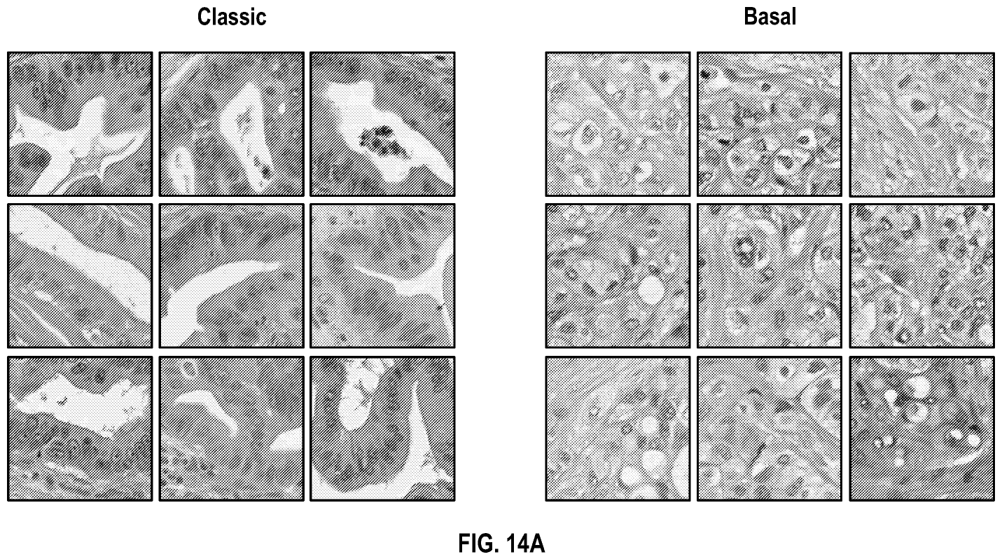

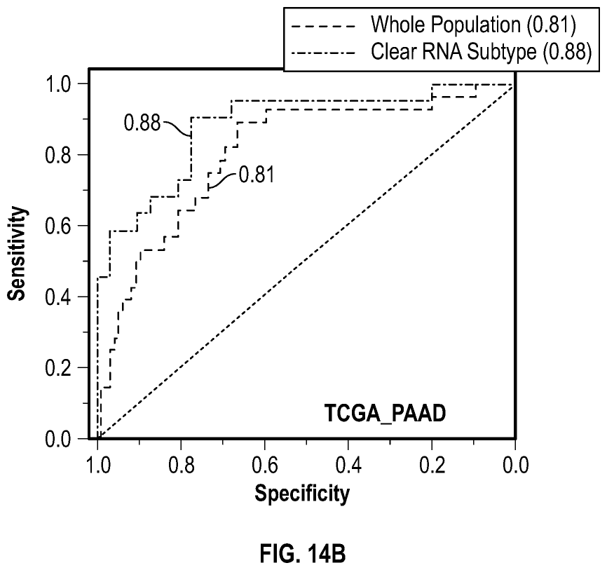

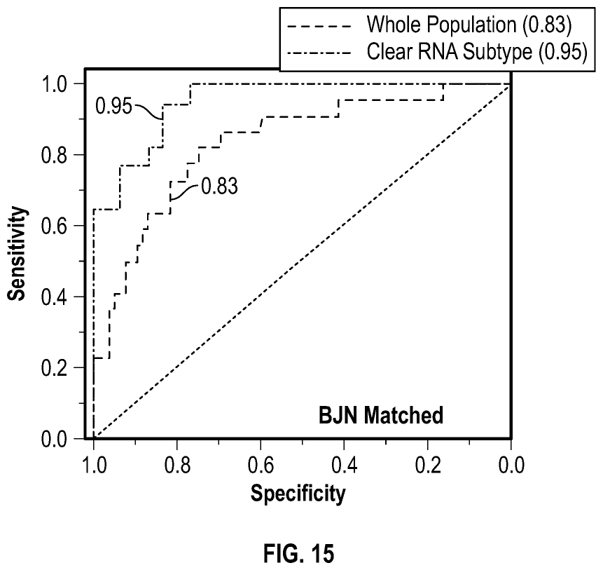

Transcriptomic data were available for all cases (from the same lesion but spatially unmatched areas) and used to train the second step of the model building on predicted neoplastic areas to determine the PurIST-RNA defined basal-like/classical (B/C) subtype (PACpAInt-B/C). Histologically, although these tumors present a highly diverse morphology, the patterns could be grouped in two sets corresponding to the basal-like or classical subtype (FIG. 13A). When applied to the validation cohorts sampled for the RNAseq in a similar manner as the discovery cohort, i.e spatially unmatched, the AUC for the prediction with PACpAInt-B/C of the subtypes was 0.86 [0.79-0.94] and 0.81 [0.71-0.90] in the BJN cohort (FIG. 13B) and the TCGA_PAAD respectively (FIG. 14A-14B). The performance of the model was comparable on a cohort with spatially matched histological and molecular areas (AUC=0.83 [0.73-0.93]) (FIG. 15).

Given the previously described intratumor heterogeneity, the analysis was subsequently restricted to the 50% of cases that had the clearest molecular subtype (low molecular heterogeneity) and showed that the performance improved substantially (AUC of 0.91 [0.84-0.98] and 0.88 [0.79-0.98] in the BJN cohort (see FIG. 13B. “clear RNA subtype”) and the TCGA_PAAD (see FIG. 14B. (“clear RNA subtype”), respectively. This was particularly significant within the matched validation cohort (see FIG. 15. AUC=0.95 [0.90-1.0]) highlighting the limitations of a binary classification in highly heterogeneous tumors.

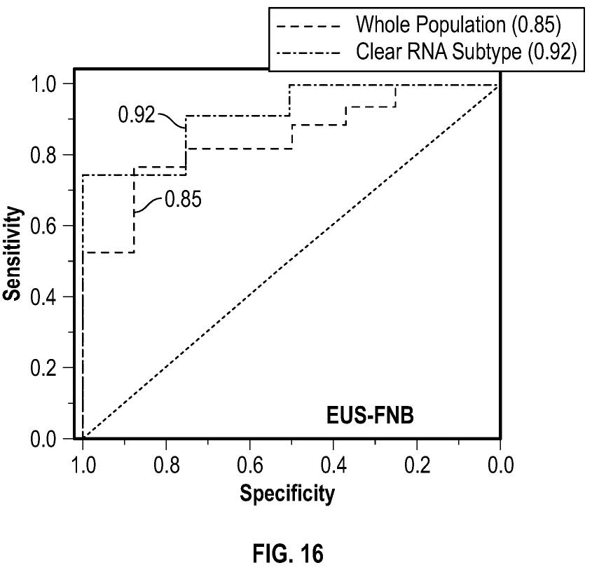

Because most patients are diagnosed at the metastatic stage on liver biopsies, the PACpAInt-B/C model was tested on 25 fine needle liver biopsies (EUS-FNB) with matched RNAseq data. The model performed equally (AUC=0.85 [0.69−1.0]) and like the surgical specimens, it improved in cases with a clear molecular subtype (AUC=0.92 [0.77−1.0], (FIG. 16).

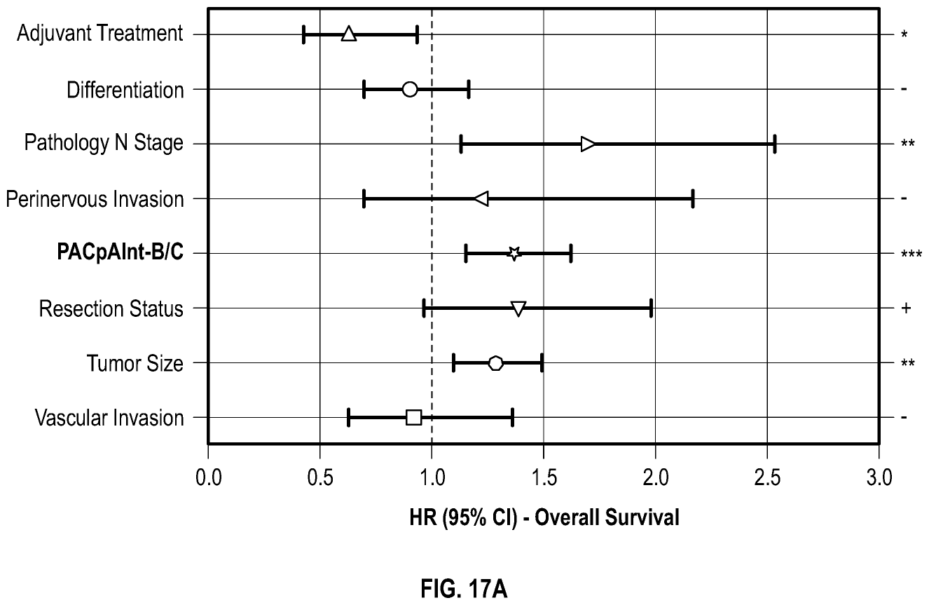

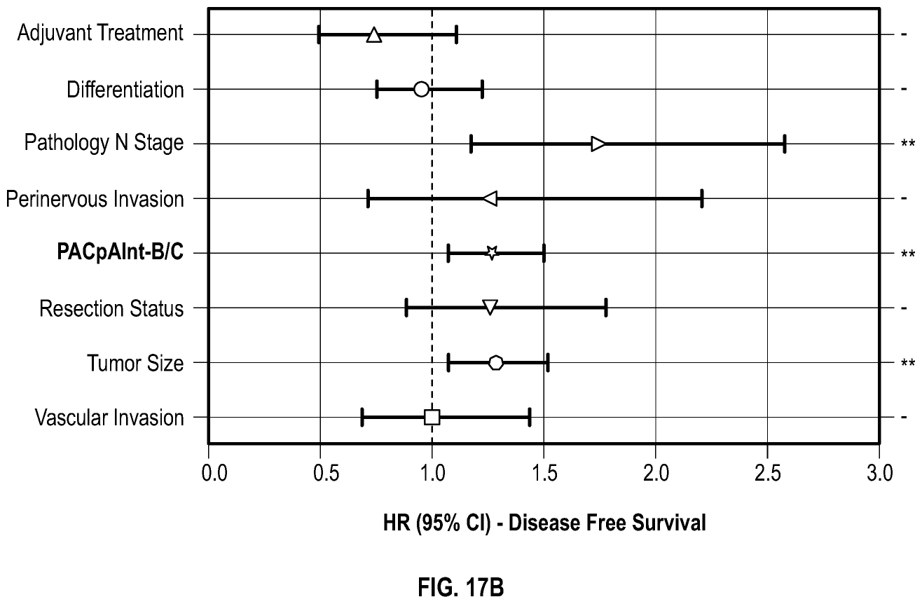

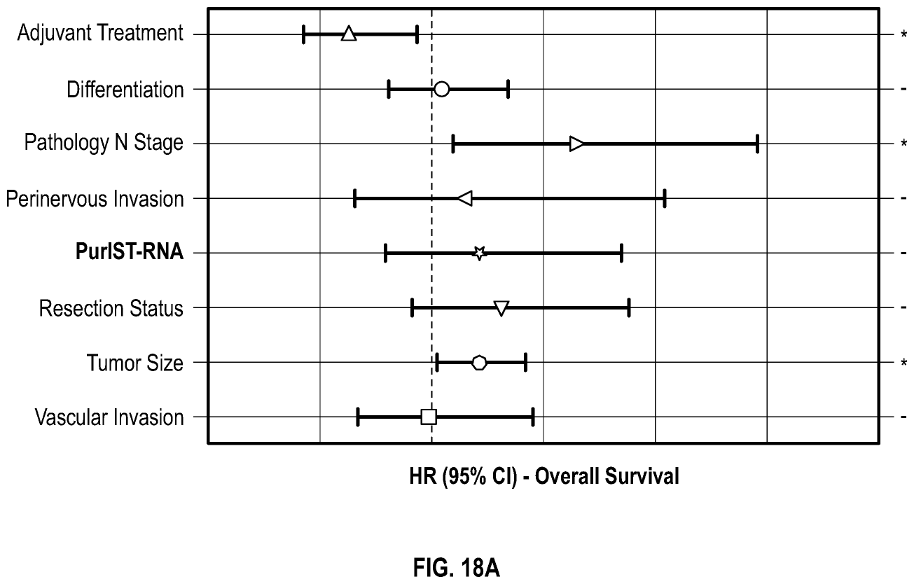

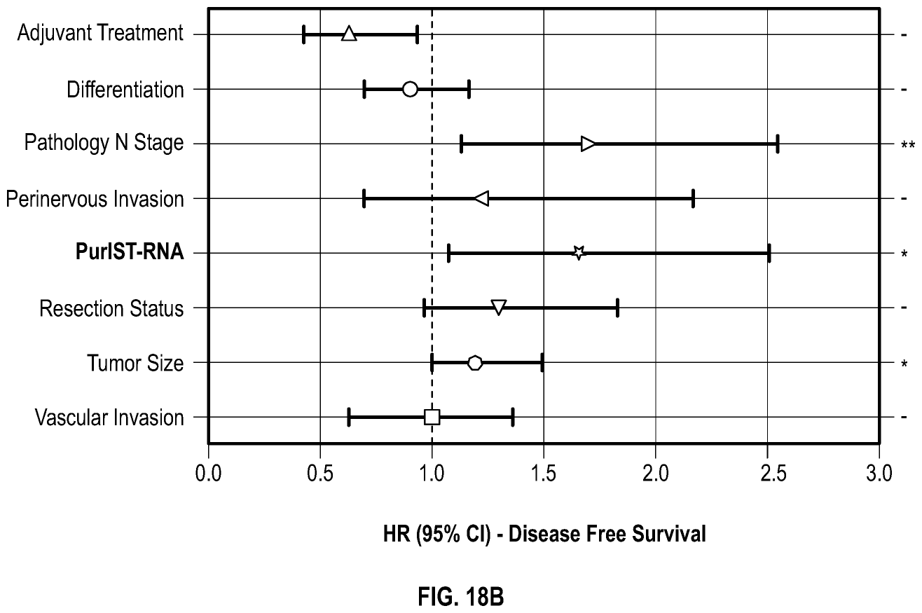

In the BJN validation cohort, in multivariate analyses the PACpAInt-B/C together with the N stage and the tumor size had a strong independent prognostic value on both overall survival (OS; FIG. 17A) and disease-free survival (DFS; FIG. 17B) (HR=1.37 [1.16−1.62] p<0.001 and HR=1.27 [1.08−1.49] p=0.003 and respectively) (see also Table 2). In contrast, the PurIST-RNA classification had an independent prognostic value on DFS but not on OS (FIG. 18A and FIG. 18D; see also Table 3).

TABLE 2
Table 2: PACpAInt-B/C in BJN validation cohort

| | Overall Survival | | Disease-Free Survival | |
|---|---|---|---|---|
| Covariate | HR (CI 95%) | p-value | HR (CI 95%) | p-value |
| Differentiation | 0.90 (0.70-1.16) | 0.432849 | 0.96 (0.76-1.22) | 0.757666 |
| Vascular Invasion | 0.93 (0.63-1.36) | 0.696855 | 1.00 (0.69-1.44) | 0.980046 |
| Perinervous Invasion | 1.23 (0.70-2.17) | 0.475994 | 1.26 (0.72-2.20) | 0.423357 |
| Tumor Size | 1.29 (1.10-1.50) | 0.001313 | 1.29 (1.08-1.53) | 0.004102 |
| Pathology N Stage | 1.70 (1.14-2.53) | 0.009221 | 1.74 (1.17-2.58) | 0.005754 |
| Resection Status | 1.39 (0.97-2.00) | 0.072018 | 1.26 (0.89-1.78) | 0.196192 |
| Adjuvant Treatment | 0.63 (0.43-0.93) | 0.020707 | 0.74 (0.50-1.11) | 0.147249 |
| PACpAInt-B/C | 1.37 (1.16-1.62) | 0.000218 | 1.27 (1.08-1.49) | 0.003158 |

TABLE 3
Table 3: PurIST-RNA in BJN validation cohort

| | Overall Survival | | Disease-Free Survival | |
|---|---|---|---|---|
| Covariate | HR (CI 95%) | p-value | HR (CI 95%) | p-value |
| Differentiation | 1.04 (0.82-1.33) | 0.741824 | 0.99 (0.78-1.25) | 0.903024 |
| Vascular Invasion | 0.99 (0.67-1.45) | 0.947547 | 1.00 (0.69-1.45) | 0.997119 |
| Perinervous Invasion | 1.16 (0.66-2.05) | 0.610294 | 1.20 (0.69-2.11) | 0.518107 |
| Tumor Size | 1.21 (1.03-1.41) | 0.020576 | 1.20 (1.02-1.43) | 0.032051 |
| Pathology N Stage | 1.64 (1.10-2.46) | 0.015744 | 1.73 (1.16-2.57) | 0.007084 |
| Resection Status | 1.31 (0.91-1.88) | 0.141978 | 1.27 (0.90-1.81) | 0.175429 |
| Adjuvant Treatment | 0.64 (0.43-0.94) | 0.024102 | 0.72 (0.48-1.07) | 0.107657 |
| PurIST-RNA | 1.21 (0.79-1.85) | 0.375660 | 1.69 (1.13-2.52) | 0.010171 |

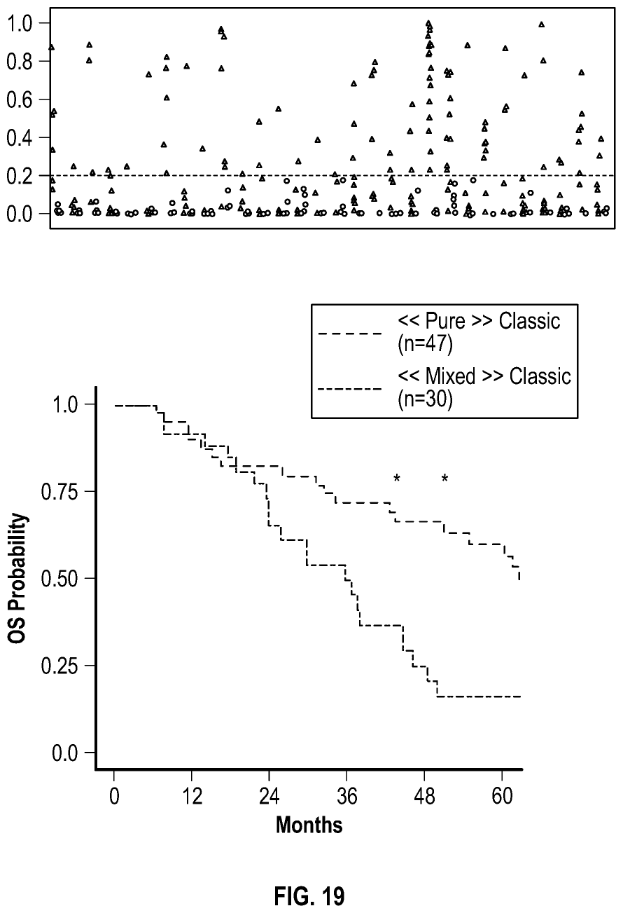

It has been previously shown that tumor cells may harbor distinct morphology from slide to slide within a case. This may be particularly meaningful in RNA-defined classical tumors that may harbor less differentiated areas that could impact the prognosis. Yet, this heterogeneity is cumbersome and difficult to properly assess and quantify. To address this question, we selected the RNA-defined classical cases in our matched validation cohort (n=77/97) and analyzed all the tumor slides with the PACpAInt-B/C model (mean number of slides/cases=9) (FIG. 19). 47 (61%) cases were homogeneous with all the slides being clearly predicted classical while in the rest of the cases the prediction differed from one slide to another clearly suggesting an important morphological and probably molecular heterogeneity. DFS and OS of heterogeneous cases were shorter (median survival of 35 vs 15 months, p=0.08 and of 64 vs 36 months, p=0.002 respectively) highlighting the clinical impact of tumor heterogeneity (FIG. 19).

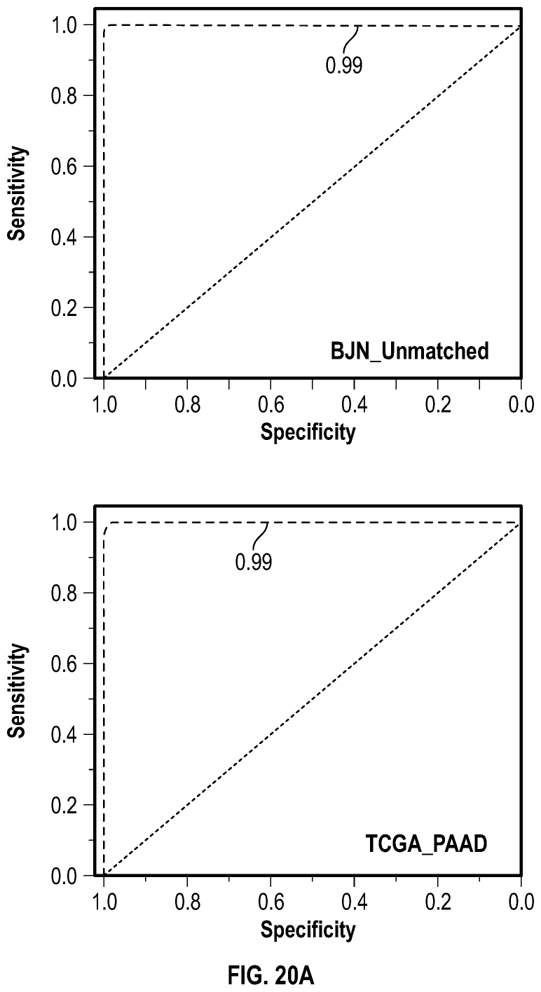

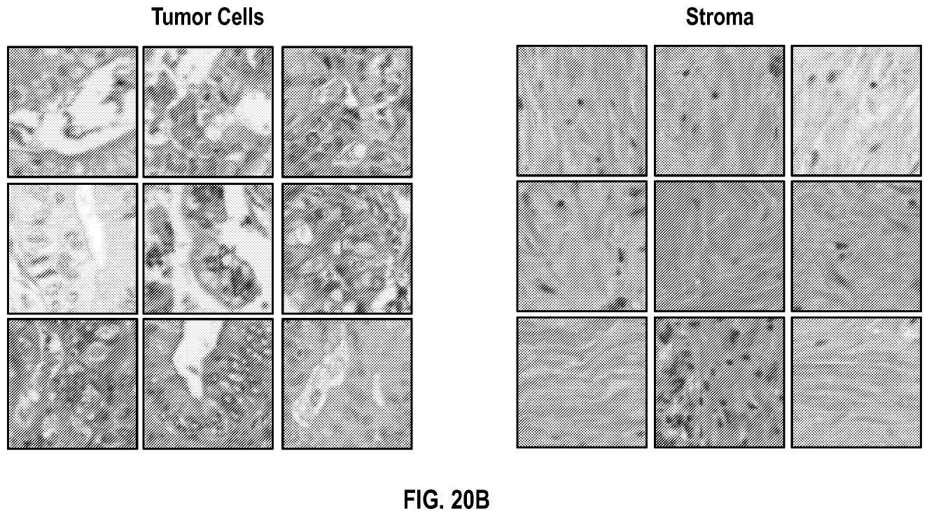

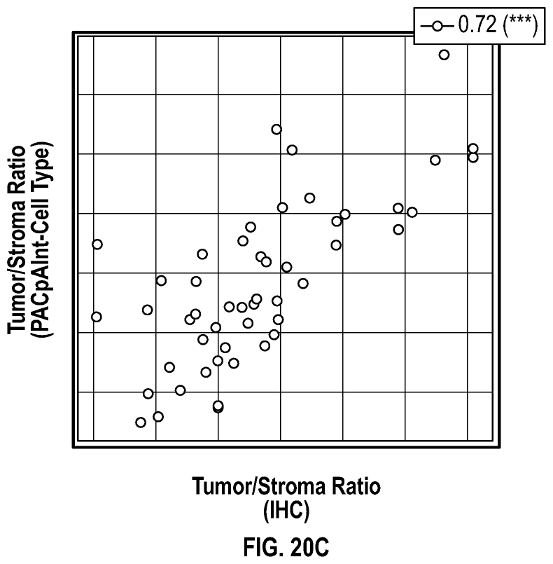

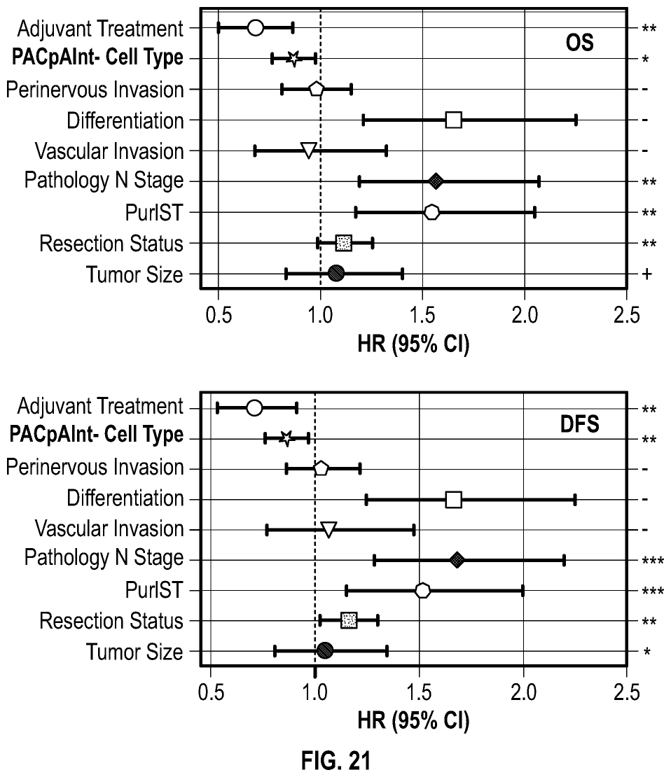

While this whole slide tumor cell-centered approach already proved its potential, it is limited to slide-to-slide heterogeneity and does not take into account the stroma, an important part of the PAC biology. PACpAInt was therefore further trained to differentiate tumor cells from stroma within the neoplastic area at the resolution of 112 micron-wide squares called tiles (PACpAInt-cell type model). The performance of the model was good with an AUC of 0.99 in the two validation cohorts (FIG. 20A, FIG. 20B). The amount of stroma quantified by immunohistochemistry and/or special stains was reported to be associated with the prognosis on small cohorts. This model was validated on a subset of cases (n=50) for which the tumor cell/stroma ratio was digitally computed using a pan-cytokeratin immunohistochemistry (Pearson's R=0.72, p<0.001) (FIG. 20C). Using this model on the whole cohort (n=451), it was confirmed that a high amount of stroma was independently associated with a better prognosis (HR=0.86 [0.76-0.96], p=0.01 and HR=0.87 [0.77-0.98], p=0.02 for DFS and OS respectively) (FIG. 21 and Table 4).

TABLE 4
Table 4: PACpAInt-Cell type prognostic value

| | Overall Survival | | Disease-Free Survival | |
|---|---|---|---|---|
| Covariate | HR (CI 95%) | p-value | HR (CI 95%) | p-value |
| Differentiation | 0.97 (0.81-1.16) | 0.731474 | 1.02 (0.87-1.21) | 0.779387 |
| Vascular Invasion | 1.08 (0.83-1.40) | 0.558079 | 1.04 (0.81-1.34) | 0.745010 |
| Perinervous Invasion | 0.95 (0.68-1.32) | 0.763570 | 1.06 (0.77-1.47) | 0.721504 |
| Tumor Size | 1.12 (1.00-1.26) | 0.055174 | 1.15 (1.02-1.30) | 0.019459 |
| Pathology N Stage | 1.66 (1.22-2.25) | 0.001374 | 1.67 (1.24-2.25) | 0.000667 |
| Resection Status | 1.55 (1.18-2.05) | 0.001951 | 1.52 (1.15-1.99) | 0.002869 |
| Adjuvant Treatment | 0.66 (0.51-0.87) | 0.002713 | 0.70 (0.53-0.91) | 0.007784 |
| PurIST | 1.57 (1.19-2.07) | 0.001470 | 1.68 (1.28-2.19) | 0.000153 |
| PACpAInt-Cell type | 0.87 (0.77-0.98) | 0.018986 | 0.86 (0.76-0.96) | 0.008231 |

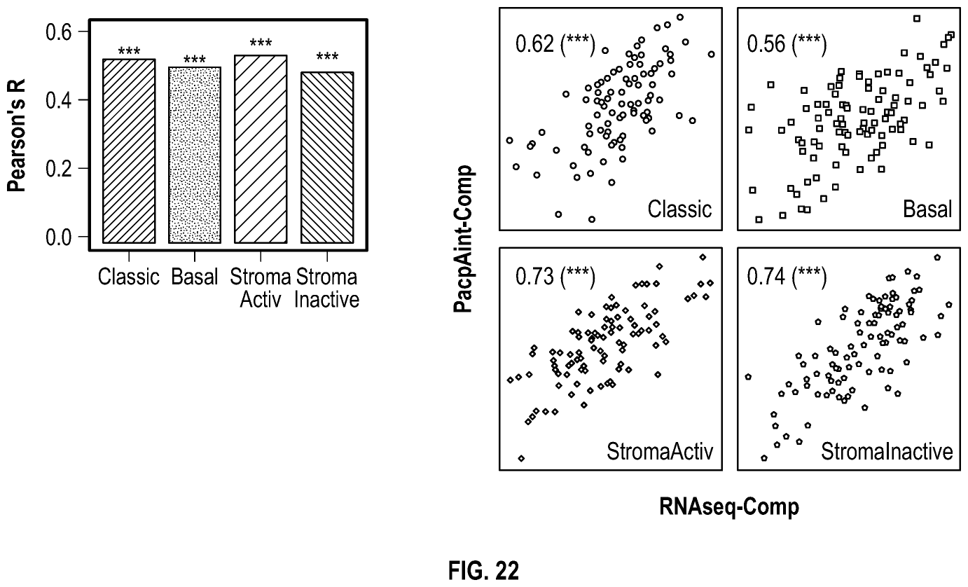

Previously developed approaches can quantify the various tumor cells (classical/basal-like) and stroma (active/inactive) phenotypes based on transcriptome profile. We therefore further trained PACpAInt to recognize the four transcriptomic components at the whole slide level using a deep learning architecture enabling a tile level inference (PACpAInt-Comp) (FIG. 11C). The correlation between the transcriptomic components and PACpAInt-Comp was highly significant in the unmatched validation cohort and further improved in the spatially matched validation cohort (+17 Pearson's R in average for the four components) (FIG. 22).

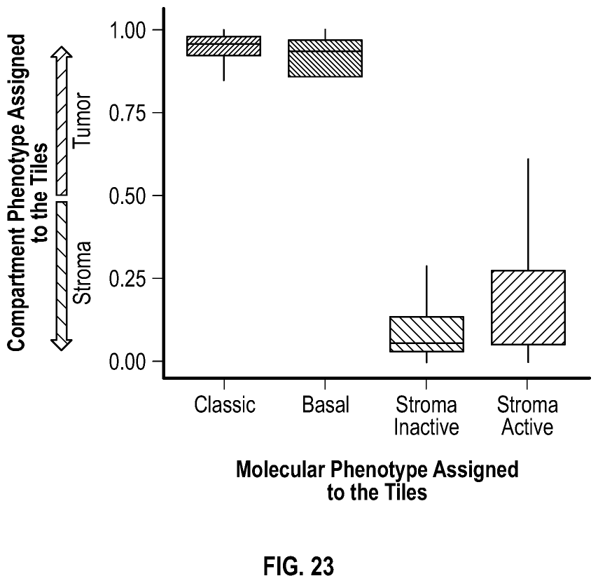

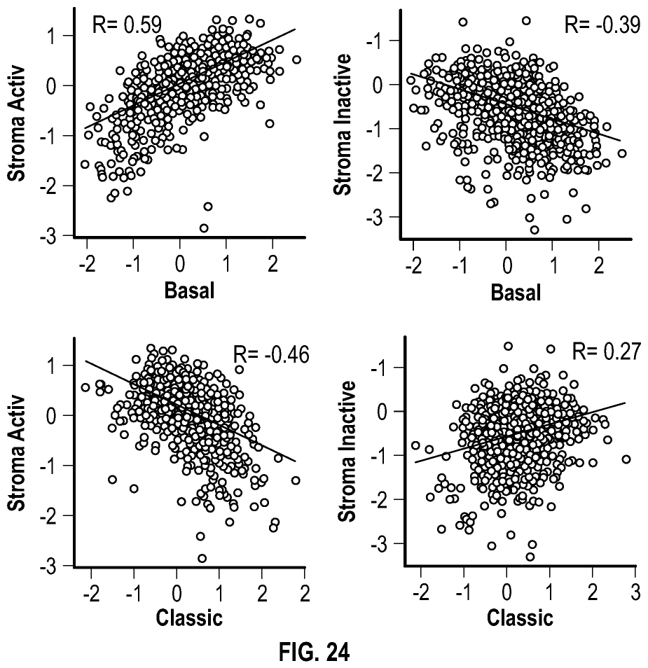

In order to validate PACpAInt-Comp local accuracy in predicting phenotypes at the tile level, the concordance between the model (classical/basal-like on tumor cell tiles and active/inactive on stroma tiles) and the scoring of two expert pathologists in PAC, was first assessed, and found to be excellent (concordance=100% and 99.2% for tumor components and 99.2% and 99.4% for stroma components). In addition, tiles with high classical/basal-like component scores were tiles with a high tumor cell score and tiles with high active/inactive stroma scores were tiles with a high stroma score (FIG. 23). To complete the local validation of PACpAInt-Comp, slides were stained with GATA6 and KRT17 antibodies, two recognized markers of classical and basal-like phenotype respectively. Stained tumor areas were selected, and the tiles within were scored for the tumor cell components. Prediction of both tumor components was good with an AUC of 0.87 for the basal component and 0.75 for the classical component, discriminating KRT17+ from GATA6+ areas (classical). PACpAInt-Comp allowed us to study the relationship between components demonstrating a strong association between active stroma and basal-like rather than classical (FIG. 24). In a multivariate Cox model, the use of the PACpAInt-Comp local components significantly improved the prognostic prediction (+4 c-index, p=0.007 and +3 c-index, p=0.008 for OS and DFS respectively) (Table 5).

TABLE 5
Table 5: Cox Model with Tile Scores

| Covariates | Overall Survival | Disease-Free Survival |
|---|---|---|
| Clinical variables | 0.63 | 0.62 |
| Clinical variables + PurIST | 0.62 | 0.63 |
| Clinical variables + tile scores | 0.67 | 0.65 |

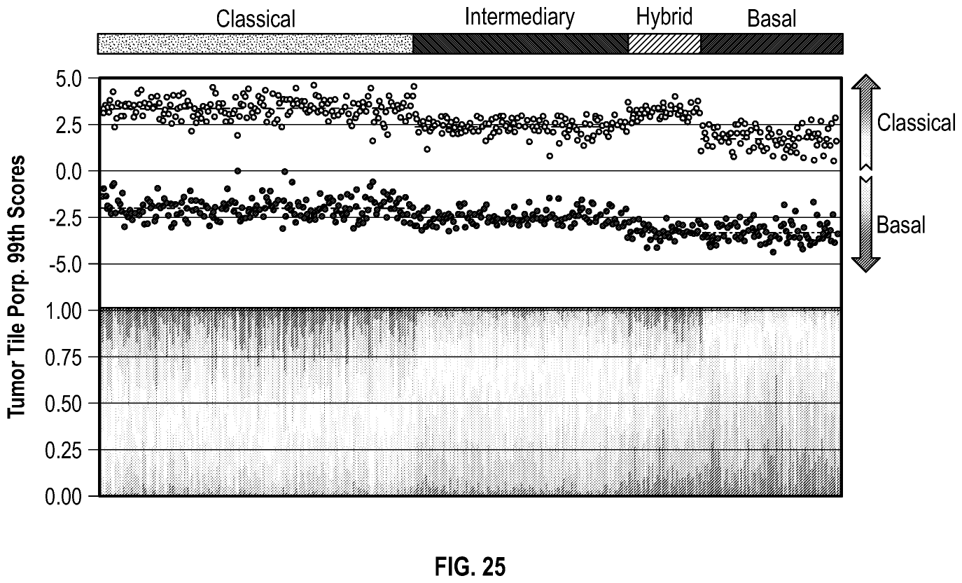

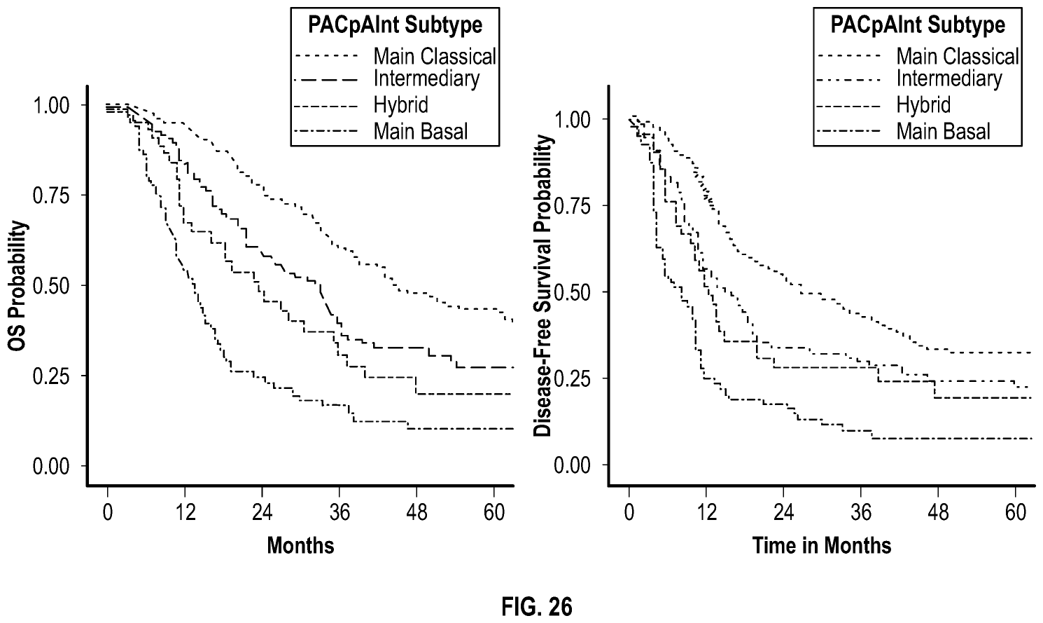

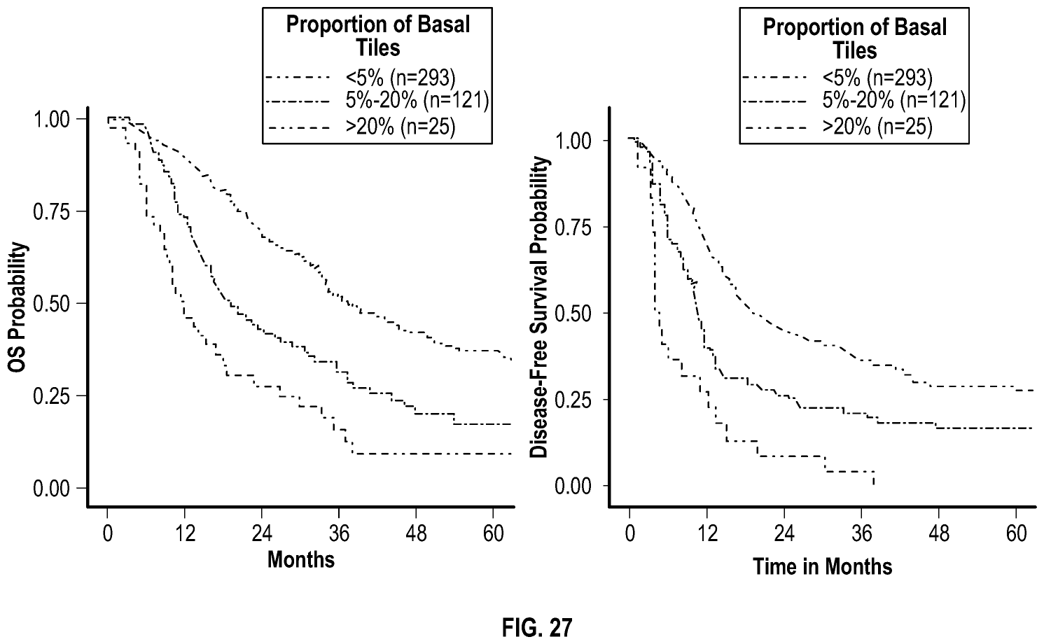

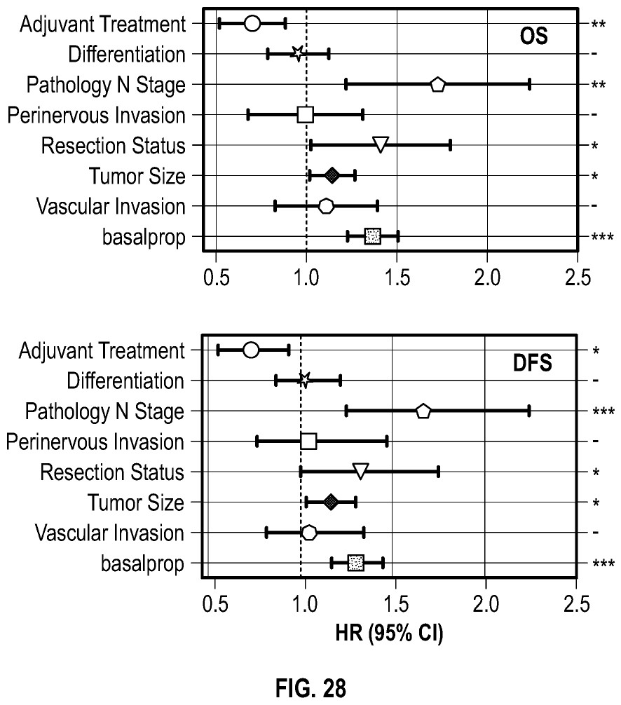

To assess the prognostic impact of intratumor heterogeneity, a total of 6.3 million tumor tiles encompassing 451 patients were phenotyped, i.e. assigning a score for each tumor/stroma component measuring its level of intensity. The distribution of the Basal-like and Classical scores showed that only 60% of tumors had a distinguishable main subtype (Classical 41%, Basal-like 19%). The remaining could be divided into an infrequent hybrid subtype (10%) defined by the coexistence of both well differentiated Basal-like and Classical tumor cells, and an intermediary subtype (30%) for which most tumor cells could not be clearly assigned to any of the two subtypes (FIG. 25). The latter two subtypes had an intermediate prognosis, for both overall survival (FIG. 26, left panel) and disease-free survival (FIG. 26, right panel). Single-cell analysis (12 cases) has shown that most tumors may be composed of Basal-like tumor cells. The application of PACpAInt-Comp to 451 cases reveals that 71% of tumors present a detectable fraction of highly Basal-like tumor cells. The overall proportion of Basal-like cells was prognostic, with worsen prognosis starting at 5% highly Basal-like tumor cells, for both overall survival (FIG. 27, left panel) and disease-free survival (FIG. 27, right panel). This basal proportion was in addition an independent poor prognostic factor on overall and disease free survival (FIG. 28 and Table 6).

TABLE 6
Table 6: Basal proportion AI - 450

| | Overall Survival | | Disease-Free Survival | |
|---|---|---|---|---|
| Covariate | HR (CI 95%) | p-value | HR (CI 95%) | p-value |
| Differentiation | 0.94 (0.79-1.12) | 0.494463 | 1.03 (0.87-1.22) | 0.734589 |
| Vascular Invasion | 1.07 (0.83-1.39) | 0.598309 | 1.05 (0.82-1.35) | 0.709357 |
| Perinervous Invasion | 0.94 (0.68-1.31) | 0.734965 | 1.06 (0.77-1.47) | 0.716473 |
| Tumor Size | 1.13 (1.01-1.26) | 0.032572 | 1.16 (1.03-1.30) | 0.012455 |
| Pathology N Stage | 1.65 (1.22-2.24) | 0.001172 | 1.67 (1.25-2.24) | 0.000521 |
| Resection Status | 1.36 (1.02-1.79) | 0.033156 | 1.32 (1.00-1.75) | 0.047713 |
| Adjuvant Treatment | 0.67 (0.52-0.88) | 0.004106 | 0.72 (0.55-0.94) | 0.015794 |
| Basal proportion | 1.36 (1.22-1.50) | 0.000000 | 1.30 (1.17-1.45) | 0.000001 |

The foregoing study demonstrates that the deep learning approach described herein is able to predict PAC molecular subtypes of both tumor and stromal cells on routine pathology slides. The interpretable deep learning design allows molecular signatures defined on whole tumors to be translated into morphology-based spatialized cell phenotyping for comprehensive intra-tumoral heterogeneity analyses.

The training cohort included slides from different centers over a long period of time with different staining protocols, to encompass a wide variability in staining. The robustness of the model is evidenced by the good performance achieved on the validation cohorts, and the multicentric TCGA_PAAD cohort. Moreover, the model performed well on liver biopsies, the most common diagnostic sample for PAC diagnosis. In addition to the binary classification of tumor cells that could help decide treatment instantly, without a lengthy and costly RNAseq analysis, the model could be deployed in clinical trials to stratify patients. In addition it could detect the remaining tumor cells after neoadjuvant treatment, paving the way for a standardized regression score that could also be used in trials to adjust the adjuvant therapy. The models described herein also make it possible to assess PAC intra-tumor heterogeneity at a level never explored before. These results provide the first clear picture of PAC intratumor heterogeneity, showing that almost a third of the tumors are probably halfway between the classical and the basal subtypes. In addition, the data shows that a minor basal-like component, that would be ignored by binary classifications, has a strong prognostic implication. This study also demonstrates that the stromal compartment can be rapidly subtyped, paving the way for patient stratification in drug targeting trials. Accordingly, AI-based PAC subtyping could be deployed to guide patient stratification based on powerful molecular criteria.

### Methods

The following methods are representative of those that were used in the Examples set forth herein.

Datasets description. The discovery set used to develop our models is a multicentric cohort of 202 patients treated in Saint-Antoine University Hospital. Pitié-Salpêtrière University Hospital or Ambroise Paré University Hospital, between September 1996 and December 2010. At least two hematoxylin-eosin+/−safran (HES) slides from surgical specimens were available for each patient, corresponding to a total of 424 slides. BJN_unmatched. BJN_matched are two independent validation cohorts of patients treated at Beaujon University Hospital between September 1996 and January 2014. BJN_unmatched consists of 304 HES slides of surgical resection specimens from 148 patients. For the BJN_matched cohort, all slides of the tumor specimens were digitized, corresponding to a total of 909 HES slides for 100 patients. EUS-FNB is a third independent validation cohort of endoscopy ultrasound fine needle biopsies from liver metastasis of 25 patients (one biopsy per patient) treated at Beaujon University Hospital between 2013 and 2020. TCGA_PAAD is a multicentric independent validation cohort of 134 hematoxylin-eosin (H&E) slides (126 cases) from a public dataset of the TCGA database. Inclusion criteria for all cohorts were as follows: unequivocal diagnosis of PAC, available histological slides of formalin-fixed, paraffin-embedded material, available follow-up and molecular information, absence of metastasis at diagnosis. This led to the exclusion of 34 slides from the TCGA that had either no tumor cell on the slide, or were from frozen examinations.

Transcriptome profiling and molecular subtyping. The discovery cohort corresponds to 202 resected tumors from the Puleo et al. study (Puleo et al., Gastroenterology (2018). 155:1999-2013) which were profiled using U219 Affymetrix microarrays. For the BJN_unmatched cohort. RNA was extracted from a 0.8 mm diameter core sampled from a tumor-enriched zone. In most cases, the RNA was not extracted from the same block that was used to generate the HES slides. For the BJN_matched and EUS_FNB series. RNA was extracted after manual microdissection to remove contaminating normal liver or pancreatic tissue. For these cohorts, the RNA was extracted from exactly the same area as the analysed HES. In addition, for the BJN_matched cohort, all the other tumor slides were also analyzed by PACpAInt. For the BJN cohorts. DNA/RNA was extracted using the ALLPrep FFPE tissue kit (Qiagen. Venlo. The Netherlands) following the manufacturer's instructions and sequenced using 3′ RNAseq (Lexogene Quantseq 3′). RNAseq reads were mapped using STAR (REF star) and genes were quantified using FeatureCount. Gene-counts were Upper Quartile-normalized and logged. PurIST was applied to both microarray and RNAseq profiles resulting in a class label for each sample. The tumor and stroma components were applied to both microarray and RNAseq profiles resulting in a continuous score for each component in each sample. For each dataset, the difference between the scaled basal and classic component scores were computed and samples that had a difference above the median were considered to have a clear RNA subtype.

Preprocessing of whole-slide images. The application of deep-learning algorithms to histological data is a challenging problem, particularly due to the high dimensionality of the data (up to 100,000×100,000 pixels for a single whole-slide image) and the small size of available datasets. Therefore a preprocessing pipeline composed of multiple steps was used to reduce dimensionality and clean the data. This pipeline includes detecting the tissue on the WSI: a U-Net neural network is used to segment part of the image that contains matter, and discard artifacts such as blur, pen marker etc., as well as the background. An additional step includes tiling the slide into smaller images, called “tiles”, of 112×112 μm (224×224 pixels). For the Example provided herein, at least 20% of the tile must have been detected as foreground by the U-Net model to be considered as a tile of matter. A maximum of 8000 such tiles are then uniformly sampled from each slide. An additional step includes extracting features from each tile: 2,048 relevant features were extracted using a self-supervised model, MoCo v2, using the approach proposed by Dehaene et al., “Self-Supervision Closes the Gap Between Weak and Strong Supervision in Histology” (2020), arXiv (https://arxiv.org/pdf/2012.03583). At the end of this preprocessing pipeline, each slide was represented by a matrix of size (nₜᵢₗₑₛ, 2048).

Neoplastic and cell type Prediction. PACpAInt neoplastic prediction model (PACpAInt-Neo) was trained at the tile level, based on neoplastic annotations of 433 slides from the discovery cohort provided by two expert pathologists, which corresponds to a total of 9,886,596 tiles. WSI preprocessing described in section “Preprocessing of whole-slide images” was used to obtain 2,048 features for each tile. PACpAInt-Neo consists of a multi-layer perceptron with a single layer of 128 hidden neurons, followed by ReLU activation. The model was validated on regions annotated by two pathologists of slides of the cohort BJN and TCGA_PAAD (10) slides for each cohort). PACpAInt cell type segmentation model (PACpAInt-Cell type) had the same architecture as PACpAInt-Neo, and was trained on 81 slides from the discovery cohort, for which stroma and tumor cells were annotated. PACpAInt-Cell type was also validated on BJN and TCGA-PAAD (10 slides for each cohort).

Molecular Prediction. PACpAInt-B/C and PACpAInt-Comp are two deep learning models that were trained on the discovery cohort to predict respectively PurIST basal classification and the molecular components Classic. Basal. StromaActiv. StromaInactive. The two models use the same WSI preprocessing pipeline described in section “Preprocessing whole-slide images”. PACpAInt-Neo was further applied on the tile features in order to select only tiles in neoplastic regions (e.g., tiles with a neoplastic prediction score larger than 0.5). PACpAInt-B/C architecture was similar to the one proposed by Ilse et al., “Attention-based deep multiple instance learning”. International conference on machine learning. PMLR (2018): A linear layer with 128 neurons was applied to the embedding followed by a Gated Attention layer with 128 hidden neurons. A MLP with 128 and 64 hidden neurons and ReLU activations was then applied to the results. A final Sigmoid activation was applied to the output to obtain a score between 0 and 1, which represents the probability of the slide to be Basal or Classic. PACpAInt-B/C was trained with the binary cross entropy as loss function. PACpAInt-Compo was inspired by the WELDON algorithm: A set of one dimensional embeddings was computed for the tile features using a multi-layer perceptron (MLP) with 128 hidden neurons followed by 4 neurons and ReLU activation (Durand et al., WELDON: Weakly Supervised Learning of Deep Convolutional Neural Networks. 29th IEEE Conference on Computer Vision and Pattern Recognition (CVPR 2016). June 2016. Las Vegas, NV. United States). For each channel output of the MLP. R=100 top and bottom scores were selected and averaged, so that the model's output is a vector of size 4, corresponding to the predicted values of each molecular component. PACpAInt-Compo was trained with the mean squared error as loss function.

Spatial Validation. To validate locally the accuracy of PACpAInt-Comp to predict Classic and Basal, GATA6 and KRT17 IHCs were performed on 12 slides of BJN matched. Tile scores for Classic and Basal components were analyzed in regions defined as being classical/basal by the IHCs. Two expert pathologists also analyzed tiles predicted to be classical or basal (n=500) and tiles predicted to be stroma active or inactive (n=500)), blinded to scores associated with each tile.

Performance assessment and statistical methods. The area under the receiver operating characteristic curve (AUC) was used to quantify the capability of the model to distinguish Classic from Basal tumors, as assessed by the PurIST method. The same metric was used to assess the performance of PACpAInt-Neo to distinguish normal from neoplastic regions, and of PACpAInt-Cell to distinguish stroma from epithelial tumor cells. Delong's method was used to compute confidence intervals at 95% confidence level (DeLong, et al., “Comparing the areas under two or more correlated receiver operating characteristic curves: a nonparametric approach.” Biometrics (1988): 837-845). Pearson's correlation was used to assess the performance of the PACpAInt-Comp model to predict the molecular components. Survival analyses were performed with uni- and multivariate Cox proportional hazards models implemented in the lifelines package of Python (Davidson-Pilon et al., CamDavidsonPilon/lifelines: v0.22.9 (Version v0.22.9). Zenodo. http://doi.org/10.5281/zenodo.3523175, 30 Oct. 2019). Log-rank tests were used to compare survival distributions between population subgroups. Survcomp R package was used to compare c-indexes (M. S. Schroder et al., “Survcomp: an R/Bioconductor package for performance assessment and comparison of survival models.” Bioinformatics (2011), 27(22), 3206-3208). All tests were two-tailed, and P values<0.05 were considered statistically significant.

Intratumoral heterogeneity subtypes. The 99th percentile of the basal and classical component scores defined by PACpAInt-Comp for each patient, using all available slides per patient, were computed. The difference between 99th basal and classical was then used to differentiate two groups of tumors using a mixture of gaussians, a high difference group of tumors considered as a well-differentiated as either basal or classical, and a low difference group of tumors with ambiguous basal/classic differentiation. The maximum of the basal or classical components was then used to separate further the low difference group: the high maximum group with low difference between extremes was termed hybrid given both high classical and basal differentiation, while the low maximum group with low difference between extremes was termed intermediary as no tumor cell tile reached a high level of either basal or classical differentiation.

Clinical variables used in the multivariate analysis. Clinical variables considered for multivariate analysis were common variables known to be associated with PAC prognosis: pN stage, differentiation, perineural invasion, resection status, tumor size, vascular invasion, and adjuvant treatment yes/no.

The foregoing discussion and examples merely describe some exemplary embodiments of the present invention. One skilled in the art will readily recognize from such discussion, the accompanying drawings, and the claims, that various modifications can be made without departing from the spirit and scope of the invention.

1-10. (canceled)

11. A computer-implemented method of determining a pancreatic ductal adenocarcinoma (PDA) subtype corresponding to a PDA classification scheme of a subject having PDA, comprising:
receiving a digital image of a histologic section of a PDA sample derived from the subject;
preprocessing the image to extract a set of features, wherein the preprocessing includes,
tiling the digital image into a set of tiles, and
performing a feature extraction on the set of tiles to extract a set of features from the set of tiles;
selecting a subset of tiles that represent one or more tumoral tissue segments, wherein the subset of tiles includes a subset of features; and
determining a PDA subtype for the digital image from at least the subset of features using a machine learning model, wherein the machine learning model is trained for the PDA classification scheme, and each of the PDA subtypes is a PDA subtype of the PDA classification scheme.

12. The computer-implemented model of claim 11, further comprising: computing one or more PDA molecular component scores for each tile of the subset of tiles using the machine learning model.

13. The computer implemented model of claim 11, wherein the machine learning model is further trained to compute a score for each PDA subtype of the classification scheme.

14. The computer implemented model of claim 11, wherein the PDA classification scheme is PurIST, and wherein the PDA subtypes include Classical and Basal-like.

15. The computer implemented model of claim 11, wherein the PDA classification scheme is Molecular Component profiling, and wherein the PDA molecular components include Classical, Basal, StromaActiv, and StromaInactive.

16. The computer-implemented method of claim 11, wherein the PDA classification scheme is one of a plurality of PDA classification schemes.

17. The computer-implemented method of claim 11, wherein each of the plurality of PDA classification schemes includes a plurality of possible PDA subtypes.

20. (canceled)

21. The computer-implemented method of claim 11, wherein the PDA sample is one of a primary pancreatic ductal adenocarcinoma, metastatic pancreatic ductal adenocarcinoma, or a portion thereof.

22-25. (canceled)

26. The computer-implemented method of claim 11, wherein the selecting of a subset of tiles is performed using a tumor model trained to distinguish tiles comprising tumor regions from tiles comprising normal regions.

27. The computer-implemented method of claim 26, wherein the tumor model comprises a multi-layer perceptron.

28. The computer-implemented method of claim 11, wherein feature extraction is performed using Momentum Contrast or Momentum Contrast v2.

29. The computer-implemented method of claim 11, wherein determining the PDA subtype for each of the tumoral tissue segments comprises:
performing an analysis of the subset of features extracted from the subset of tiles using the machine learning model to generate a subtype score corresponding to each tile in the subset of tiles.

30. The method of claim 29, wherein determining the PDA subtype includes computing a PurIST score at a slide level based on an analysis of the subset of features extracted from the subset of tiles.

31. The computer-implemented method of claim 11, wherein the machine learning model has been trained using a plurality of training images that comprise digital images of histologic sections of a PDA samples derived from subjects of known PDA subtype of the PDA classification scheme and the training images each include a global label indicative of the known PDA subtype.

32. (canceled)

33. The computer-implemented method of claim 29, wherein the machine learning model is a Deep Multiple Instance Learning model.

34-37. (canceled)

38. The computer-implemented method of claim 12, further comprising pooling the PDA molecular component scores corresponding to each tile in the subset of tiles, to generate a slide-level molecular component score, wherein the slide-level molecular component score is indicative of a molecular component with highest predicted score.

39. The computer-implemented method of claim 12, further comprising overlaying the digital image with information representative of the PDA molecular component scores corresponding to each tile in the subset of tiles, to generate a digital image labeled with information representative of the PDA molecular component score.

40. The computer-implemented method of claim 39, wherein the information representative of the PDA molecular component score of each tile comprises a label indicative of a molecular component with highest predicted score of the one or more tumoral tissue segments contained in the tile.

41-42. (canceled)

43. The computer-implemented method of claim 12, further comprising:
analyzing all PDA molecular component scores corresponding to a single tumor of a patient;
determining a proportion of slides of the single tumor corresponding to different PDA molecular component scores; and
generating a tumor-level PDA molecular component score based on the proportion of slides of the single tumor corresponding to different PDA molecular component scores.

44. A machine readable medium having executable instructions to cause one or more processing units to perform a method for processing a digital image of a pancreatic ductal adenocarcinoma (PAC) sample, the method comprising receiving a digital image of a PAC sample derived from a subject, applying a machine learning model to the digital image, and determining a PAC classification for the image using the machine learning model, wherein the machine learning model has been trained by processing a plurality of training images.

45-84. (canceled)
